(19) United States
(12) Patent Application Publication
(10) Pub. No.: US 2022/0412993 A1
(43) Pub. Date: Dec. 29, 2022

(54) Multi-Biomarker Prediction Models for Multiple Infection Episodes Following Blunt Trauma

(71) Applicants: The General Hospital Corporation, Boston, MA (US); University of Massachusetts, Boston, MA (US)

(72) Inventors: Laurence Rahme, Brookline, MA (US); Amy Tsurumi, Boston, MA (US); Patrick Flaherty, Amherst, MA (US); Colleen M. Ryan, Winchester, MA (US)

(21) Appl. No.: 17/781,646

(22) PCT Filed: Dec. 7, 2020
(86) PCT No.: PCT/US2020/063583

§ 371 Date: Jun. 1, 2022

Related U.S. Application Data
(60) Provisional application No. 63/089,512, filed on Oct. 8, 2020.
(60) Provisional application No. 62/943,855, filed on Dec. 5, 2019.

(51) Int. Cl. G01N 33/68

(52) U.S. Cl. CPC G01N 33/6893; G01N 2800/26; G01N 2800/60

(57) ABSTRACT

Described herein are methods that use blood biomarkers to identify an increased risk of multiple independent infection episodes (MIIE) before clinical signs of infection appear. Thus, provided herein are methods for detecting or predicting risk of developing multiple independent infection episodes (MIIE) in a subject who has experienced blunt trauma.

### CLAIM OF PRIORITY

This application claims the benefit of U.S. Provisional Application Ser. No. 62/943,855, filed on Dec. 5, 2019; and 63/089,512, filed on Oct. 8, 2020. The entire contents of the foregoing are incorporated herein by reference.

### FEDERALLY SPONSORED RESEARCH OR DEVELOPMENT

This invention was made with Government support under Grant No. GM062119 awarded by the National Institute of General Medical Sciences of the National Institutes of Health. The Government has certain rights in the invention.

### TECHNICAL FIELD

Described herein are methods that use blood biomarkers to identify increased risk of multiple independent infection episodes (MIIE) before clinical signs of infection appear.

### BACKGROUND

The high value of a tailored approaches in the care of patients is increasingly appreciated (Chaussabel, 2015; Parikh et al., 2016; Sweeney and Wong, 2016). A method to expedite the timeline of threat detection to before infection happens could yield valuable time for early prophylactic and therapeutic interventions. Moreover, the ability to identify patients at high risk for repeated infections, or infection-related morbidity and mortality, might be considered an important measure to fairly allocate resources such as medication, personal protective equipment, or another high-value scarce intervention (Massachusetts, 2020; Medicine, 2013; Medicine, 2020).

Trauma is one of the leading causes of morbidity and mortality worldwide (Heron, 2018; Krug et al., 2000). Severe trauma induces various immune-related responses acutely—it can trigger a state of immunosuppression (Islam et al., 2016; Ward, 2005), prolonged inappropriate immune response (Heffernan et al., 2012; Huber-Lang et al., 2018), leukocytosis (Paladino et al., 2010), and the elevation of specific subpopulations of myeloid cells (Cuenca et al., 2011). Among trauma patients, infections and infections-related complications contribute to substantial mortality and morbidity, and prolonged hospital stay, significantly adding to health care costs (Cole et al., 2014; Dutton et al., 2010; Glance et al., 2011; Hashmi et al., 2014). Infections and infections-related outcomes vary across individuals, suggesting the importance of considering individual patients' underlying susceptibility and the degree of immunosuppression, or inappropriate immune response experienced.

### SUMMARY

Severe trauma predisposes patients to multiple independent infection episodes (MIIE), leading to augmented morbidity and mortality. Described herein are methods to identify increased MIIE risk before clinical signs appear that are fundamentally different from existing approaches entailing infections' detection after their establishment. Applying unbiased machine learning algorithms to genome-wide transcriptome data from 128 adult blunt trauma patients' (42 MIIE cases and 85 non-cases) leukocytes collected ≤48 hours of injury and >3 days before any infection, 15-transcript and 26-transcript multi-biomarker panel models were constructed with the least absolute shrinkage and selection operator (LASSO) and Elastic Net, respectively, which accurately predicted MIIE (AUROC [95% CI]: 0.90 [0.84-0.96] and 0.92 [0.86-0.96]), and significantly outperformed clinical models. Gene Ontology and network analyses found various pathways to be relevant. External validation found the model to be generalizable. This unique precision medicine approach can be applied to a wide range of patient populations and outcomes.

Thus, provided herein are methods for detecting or predicting risk of developing multiple independent infection episodes (MIIE) in a subject who has experienced blunt trauma. The methods include providing a sample comprising blood, serum, or plasma from a subject who has experienced blunt trauma; detecting one, two, three, four, five, six, seven, eight, nine, ten, 11, 12, 13, 14, or all of the transcripts in a 15 probe set biomarker panel comprising or consisting of hepatocyte growth factor (HGF); kelch repeat and BTB domain containing 7 (KBTBD7); adenosine A3 receptor (ADORA3); ADP-ribosylation factor-like GTPase 4A (ARL4A); epiplakin 1 (EPPK1); zinc finger protein 354A (ZNF354A); SH3 and PX domains 2B (SH3PXD2B); RNase A family 1 (RNASE1), BTB domain containing 19 (BTBD19), zeta chain of T cell receptor associated protein kinase 70 kDa (ZAP70); endoplasmic reticulum aminopeptidase 2 (ERAP2); CD96 molecule (CD96); membrane metallo-endipeptidase (MME); killer cell lectin-like receptor subfamily F, member 1 (KLRF1); and/or non-coding transcript, nuclear paraspeckle assembly transcript 1 (NEAT1). The methods can include comparing the level of the transcript to a reference level, wherein the presence of: an increase in one or more of: hepatocyte growth factor (HGF); kelch repeat and BTB domain containing 7 (KBTBD7); adenosine A3 receptor (ADORA3); ADP-ribosylation factor-like GTPase 4A (ARL4A); epiplakin 1 (EPPK1); zinc finger protein 354A (ZNF354A); SH3 and PX domains 2B (SH3PXD2B); RNase A family 1 (RNASE1), or BTB domain containing 19 (BTBD19), or a decrease in one or more of: zeta chain of T cell receptor associated protein kinase 70 kDa (ZAP70); endoplasmic reticulum aminopeptidase 2 (ERAP2); CD96 molecule (CD96); membrane metallo-endipeptidase (MME); killer cell lectin-like receptor subfamily F, member 1 (KLRF1); and/or non-coding transcript, nuclear paraspeckle assembly transcript 1 (NEAT1), indicates that the subject has, or has an increased risk of developing (i.e., a risk above that of a reference cohort of subjects with blunt trauma), an infection, within the next 2, 3, 4, 5, 6, 7, 10, 12, 14, 20, 21, or 24 days. Alternatively or in addition, the methods can include calculating a score based on the transcript levels, and comparing the score to a threshold or reference score, wherein a score about the threshold or reference score indicates that the subject has, or has an increased risk of developing, MIIE In some embodiments, all of the transcripts in the 15 probe set biomarker panel are used; in some embodiments, 14 are used, excluding SH3PXD2B.

In some embodiments, the methods include determining a level of all of ZNF354A; EPPK1; RNASE1; BTBD19; ADORA3; KBTBD7; SH3PXD2B; CD96; ARL4A; ZAP70; ERAP2; HGF; KLRF1; NEAT1; and MME.

In some embodiments, the methods also include determining a level of one or more of Interleukin 1 receptor, type II (IL1R2) and mannose receptor, C type 1 (MRC1); importin 11 (IPO11); dedicator of cytokinesis 4 (DOCK4); Kruppel-like factor 9 (KLF9); nebulette (NEBL); a different probe set for MME; ribosomal protein S6 kinase, 90 kDa, polypeptide 5 (RPS6KA5); killer cell lectin-like receptor, subfamily K, member 1 (KLRK1); granzyme K (GZMK); and/or ADP-ribosylation factor-like GTPase 4C (ARL4C).

In some embodiments, the methods include determining a level of ARL4C; KLRK1; SH3PXD2B; EPPK1; ZNF354A; RNASE1; MME; BTBD19; MRC1; ADORA3; NEBL; KBTBD7; IP011; RPS6KA5; KLF9; DOCK4; IL1R2; CD96; ARL4A; ERAP2; GZMK; NEAT1; KLRF1; HGF; MME; and ZAP70.

In some embodiments, an increase in one or more of interleukin 1 receptor, type II (IL1R2); mannose receptor, C type 1 (MRC1); importin 11 (IP011); dedicator of cytokinesis 4 (DOCK4); Kruppel-like factor 9 (KLF9); and/or nebulette (NEBL), or a decrease in one or more of MME, ribosomal protein S6 kinase, 90 kDa, polypeptide 5 (RPS6KA5); killer cell lectin-like receptor, subfamily K, member 1 (KLRK1); granzyme K (GZMK); and/or ADP-ribosylation factor-like GTPase 4C (ARL4C) indicates that the subject has, or has an increased risk of developing (i.e., a risk above that of a reference cohort of subjects with blunt trauma), an infection, e.g., within the next 2, 3, 4, 5, 6, 7, 10, 12, 14, 20, 21, or 24 days.

Also provided herein are methods comprising: providing a sample comprising blood, serum, or plasma from a subject who has experienced blunt trauma; detecting a level of transcripts consisting of one, two, three, four, five, six, seven, eight, nine, ten, 11, 12, 13, 14, or all of hepatocyte growth factor (HGF); kelch repeat and BTB domain containing 7 (KBTBD7); adenosine A3 receptor (ADORA3); ADP-ribosylation factor-like GTPase 4A (ARL4A); epiplakin 1 (EPPK1); zinc finger protein 354A (ZNF354A); SH3 and PX domains 2B (SH3PXD2B); RNase A family 1 (RNASE1), BTB domain containing 19 (BTBD19), zeta chain of T cell receptor associated protein kinase 70 kDa (ZAP70); endoplasmic reticulum aminopeptidase 2 (ERAP2); CD96 molecule (CD96); membrane metallo-endipeptidase (MME); killer cell lectin-like receptor subfamily F, member 1 (KLRF1); and non-coding transcript, nuclear paraspeckle assembly transcript 1 (NEAT1).

In some embodiments, the methods include determining a level of all of ZNF354A; EPPK1; RNASE1; BTBD19; ADORA3; KBTBD7; SH3PXD2B; CD96; ARL4A; ZAP70; ERAP2; HGF; KLRF1; NEAT1; and MME.

In some embodiments, the methods include determining a level of one or more transcripts selected from the group consisting of Interleukin 1 receptor, type II (IL1R2) and mannose receptor, C type 1 (MRC1); importin 11 (IPO11); dedicator of cytokinesis 4 (DOCK4); Kruppel-like factor 9 (KLF9); nebulette (NEBL); a different probe set for MME; ribosomal protein S6 kinase, 90 kDa, polypeptide 5 (RPS6KA5); killer cell lectin-like receptor, subfamily K, member 1 (KLRK1); granzyme K (GZMK); and ADP-ribosylation factor-like GTPase 4C (ARL4C).

In some embodiments, the methods include determining a level of ARL4C; KLRK1; SH3PXD2B; EPPK1; ZNF354A; RNASE1; MME; BTBD19; MRC1; ADORA3; NEBL; KBTBD7; IP011; RPS6KA5; KLF9; DOCK4; IL1R2; CD96; ARL4A; ERAP2; GZMK; NEAT1; KLRF1; HGF; MME; and ZAP70.

In some embodiments, the methods include calculating a score based on the levels of the transcripts. In some embodiments, the score is calculated using an algorithm comprising summation or weighted summation of normalized levels of the biomarkers. In some embodiments, the score is calculated using principal components analysis (PCA) linear regression, support vector machine (SVM), decision trees, K-nearest neighbors (KNN), K-means, gradient boosting, or random forest methods. The methods can this include comparing a score to a threshold or reference score, wherein a score about the threshold or reference score indicates that the subject has a high risk of MIIE

In some embodiments, determining a level comprises using PCR-based methods (e.g., (RT-qPCR), RNA sequencing, next generation sequencing, digital gene expression analysis, or microarray analysis.

In some embodiments, the sample is obtained from the subject within 48 hours of the blunt trauma.

In some embodiments, the subject does not have clinical signs of an infection (e.g., fever, elevated white blood cell count, or other signs) when the sample is obtained.

In some embodiments, the infections include infection with a Gram positive or Gram negative bacterium, virus, or fungus, e.g., Streptococcus pneumonia, Streptococcus viridans, Staphylococcus aureus, Enterococcus species, Coagulase negative staphylococci, and Streptococcus pneumoniae and viridian, Clostridium sp.; Candida species, Enterobacter species, Acinetobacter species, Pseudomonas aeruginosa, Haemophilus influenza, Bacteroides species, Klebsiella pneumoniae, Neisseria, Proteus, Serratia marcescens, Escherichia coli, Stenotrophomonas, and Candida species.

In some embodiments, the methods include treating the subject, e.g., with a broad-spectrum antibiotic, increasing the frequency or length of monitoring of the subject for infection; implementing prophylactic measures; and/or enhancing patient nutrition. Those who are identified to be at high risk could can be treated with from increased surveillance and additional preventative measures taken early. Additional interventions for the high risk group can include increased surveillance for early mobilization and removal of lines/tubes, coating IV lines and urine catheters with antimicrobials and/or antibiotics, immunomodulatory nutrition therapies

In some embodiments, the subject is a mammal, e.g., a human.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Description of the patient population and study design. (A) Patients who were included/excluded in the study and (B) the study design.

FIG. 2. Timing of detection of microorganisms. Box plot of the median day of first infection since the injury, where white boxes indicate Gram positives, light gray boxes indicate Gram negatives, and dark gray boxes indicate fungi and unknown. Recorded species are indicated or described as not otherwise specified (NOS).

FIGS. 3A-B. Identification of probe sets with 1.5-fold change expression level comparing MIIE cases to non-cases, and pathway analysis. (A) Volcano plot using the initial 25,567 probe sets included in the analyses, where black dotted lines mark the log ₂(1.5) threshold, and black dots indicate probe sets with ≥1.5-fold change (137 probe sets), orange squares with labels mark data points corresponding to the 15 probe set panel, and blue triangles mark the additional probe sets, that together with the 15 probe sets, result in the 26 probe set panel. (B) Ingenuity pathway analyses (IPA) of the 26 probe sets that make up the comprehensive biomarker panel. Green marks downregulated and red marks upregulated probe sets. Solid lines indicate known direct interactions, and dotted lines indicate indirect interaction.

FIGS. 4A-B. Comparisons of the performance of the multi-biomarker panel versus the clinical severity models. ROC curves of the various models constructed and respective AUROC [95% CI], 10-fold cross-validation (CV) AUROC [95% CI], and p-value of total AUROC difference compared to the 15 probe set biomarker panel. (A) Various models: 15 probe set biomarker panel, 26 probe set biomarker panel, APACHE II, ISS, NISS. (B) Various combined biomarker and clinical score models: 15 probe set biomarker+APACHE, 15 probe set biomarker+ISS, 15 probe set biomarker+NISS, 26 probe set biomarker+APACHE, 26 probe set biomarker+ISS, 26 probe set biomarker+NISS.

FIGS. 5A-C. The AUROC curve when applying the biomarker panel model constructed using the Glue Grant, to the Cabrera et al. dataset as the external validation test set. For both the (A) 24 hour and (B) 72 hour time point of the Cabrera et al. dataset, our biomarker panel model conferred significant prediction, as evidenced by the AUROC [95% CI] significantly above 0.5. On the other hand, the prediction model with (C) Injury Severity Score (ISS) did not provide significant prediction.

FIG. 6. Decision tree used to define independent infection episodes from clinical and microbiological records, taking into account, the timing of infections, type of infection and pathogen isolated, related to FIG. 1. Each infection record was placed on a “waiting list” for six days if the types and modes of infection and pathogen were similar, or two days if they were not. This method allowed tabulation of the number of total independent infection episodes that is not an extension of a previously recorded infection from the time of the injury. Adapted from Yan et al, 2015. ²⁵

FIGS. 7A-D. LASSO and Elastic Net regression outputs, related to FIG. 4. (A) A plot of 10-fold CV repetition results, with dotted lines showing λₘᵢₙ and λ₁ₛₑ values, where 15 probe sets were selected. (B) LASSO coefficient profile plot of the coefficient paths, where at λ₁ₛₑ, the selected 15 probe sets have coefficients significantly different from zero. (C) 10-fold CV results and (D) coefficient profile plot for Elastic Net.

### DETAILED DESCRIPTION

Methods to identify trauma patients with particularly increased risk of infection could be advantageous for ensuring timely and appropriate delivery of preventative measures (such as early immune-modulating nutrition, microbiome modulation, early mobilization, early removal of lines/tubes, taking all transmission-based precautions), improving surveillance and promoting antibiotic stewardship to limit the emergence of multi-drug resistance bacteria, reduce toxicity to patients and decrease health care costs. Previous studies have evaluated the use of injury severity scores, such as the Acute Physiology and Chronic Health Evaluation (APACHE) II (Knaus et al., 1985) Injury Severity Score (ISS) (Baker et al., 1974) and New Injury Severity Score (NISS) (Osler et al., 2010) as predictors of infection, in addition to their intended use to predict mortality (Cheadle et al., 1989; Jamulitrat et al., 2002). Using genome-wide transcriptomic information from leukocytes provided at triage to assess the underlying susceptibility, well before the onset of infections, is expected to significantly improve the accuracy of identifying patients who are most at-risk of multiple independent infection episodes. A recent study described that employing a combination of predictors could be more effective than using a single predictor with strong statistical significance, further suggesting that multi-biomarker panels could be highly effective (Lo et al., 2015). Previous studies have utilized transcriptome data in the trauma setting to find transcripts that correlate with poor outcome (Desai et al., 2011) or sepsis (Sweeney et al., 2015). The objective of this study is distinct, as we aim to develop a method to predict multiple infections prior to classic clinical signs of infection. And thus, our approach focuses on the prevention of infections, aiming to predict the outcome before its onset, using early blood samples.

In a previous study among burn trauma patients, a blood transcriptomic multi-biomarker panel was developed for predicting multiple independent infection episodes (MIIE) outcome during the course of recovery (Yan et al., 2015). The least absolute shrinkage and selection operator (LASSO) machine learning algorithm was employed to select probe sets that together (i.e., a multi-transcriptome panel) resulted in highly accurate prediction. This model performed significantly better than those based on injury severity assessments at triage and demographic information (Yan et al., 2015). Described herein is a new approach combining the use of two algorithms, LASSO and Elastic Net regression, to investigate MIIE outcome. LASSO and Elastic Net were used to reduce the complexity of regression models, in conjunction with cross-validation to select the optimal parameter for reducing the number of predictors. These techniques are highly beneficial in cases such as transcriptome data where the number of potential predictors is extremely large, to overcome the problem of overfitting. LASSO regression reduces highly correlated predictors and selects a minimal panel of predictors, compared to Elastic Net that includes some correlated predictors. Blunt trauma patients in the multi-center Inflammation and Host Response to Injury (“Glue Grant”) cohort were investigated. This cohort enrolled a high number of patients, generated genome-wide transcriptome data, and collected data longitudinally, allowing the building of a predictive model. The present methods employ unbiased analyses of genome-wide information for the identification of patients at increased risk for MIIE before clinical signs of infection. These methods are also advantageous for providing new insights into the molecular pathways that characterize the pathophysiology underlying hypersusceptibility to infections.

The present study shows that employing novel prognostic models based on early blood transcriptome profiling following severe trauma is an effective method for identifying patients who are particularly at high risk for MIIE and thus, hypersusceptible to infections. That the transcriptome information provides much better prediction than injury severity information, argues for the importance of considering each patient's underlying susceptibility and of elucidating relevant molecular mechanisms. The comprehensive dataset we used had genome-wide transcriptome and clinical data collected longitudinally from a large number of patients, providing the opportunity to assess early susceptibility. Notably, these results suggest that by measuring the biomarkers at admission, patients at increased risk for MBE can be identified before any clinical signs of infection appear. The biomarker panel models used herein had particularly high specificity and NPV measures, while also exerting good sensitivity and PPV. Moreover, when applied to an external validation cohort, the models still performed. On the other hand, none of the injury severity scores often used at triage in the trauma setting were effective in predicting the MIIE

Application of both LASSO and Elastic Net regression methods allowed the construction of a highly predictive model from a minimal set of predictors. The LASSO approach selects a stringent set of predictors with less redundancy, which is advantageous in the clinical setting, where a device requiring less measurements is more practical and easier to implement. The Elastic Net approach that allows for correlated predictors to be selected found additional transcripts for a more comprehensive discovery of biological mechanisms. The probe sets selected consisted of transcripts with GO terms relevant to infections and signaling pertinent to oncogenesis and cancer progression.

In the present study, HGF was the transcript showing the highest upregulation among MIIE patient blood and in both the 15 probe set and 26 probe set panels. HGF and Met expression levels have been suggested as a putative biomarker for monitoring infections, as it is well-established that the HGF-Met signaling pathway deregulation promotes the growth and invasion by various pathogens (Imamura and Matsumoto, 2017). Another upregulated transcript, ADORA3, has also been implicated in the clinical setting, and agonists have been developed and shown to induce anti-inflammatory effects by altering the Wnt and NF-κB pathways. As such, the agonists are considered for purposes of treating cancers, and inflammatory diseases such as rheumatoid arthritis and psoriasis (Fishman et al., 2012). NEATI is a non-coding RNA that is shown to colocalize with MALAT1, a long non-coding RNA often associated with metastatic cancer, at many genomic sites to transcriptionally regulate target genes (West et al., 2014). CD96 is highly expressed in T and NK cells and well-established to be a regulator of immune responses during infection and cancer (Georgiev et al., 2018).

Elastic Net selected two probe sets corresponding to WE, providing further support for its importance in MIIE outcome. Studies on its molecular mechanisms and clinical use of inhibitors to its protein product, Neprilysin has been conducted widely, including in Alzheimer's, heart failures, hypertension, and renal diseases (Riddell and Vader, 2017). Our study suggests that its potential role in immunity among patients warrants further investigation. KLRK1 and KLRF1, both killer cell lectin-like receptor subfamily members, were found by Elastic Net, providing evidence of their relevance in infections in the blunt trauma setting. These receptors are abundant on NK cells, and it is well-established that they play crucial roles in innate immunity (Barten et al., 2001). These previous findings provide additional confidence in the relevance of our methodology. The pathway analysis found key signaling pathway components among the central nodes having extensive edges, including the major cytokines, TNF, TGFβ-1, CCLs, and CXCLs, as well as key signaling components, p38 MAPK, ERK1/2, SMAD3, and CTNNB1. These components represent the chief signaling pathways that regulate inflammation, mitogenic response, and tissue regeneration, which are also often dysregulated in cancer. Notably, the present results suggest that the TNF, TGFβ-1, and Wnt signaling pathways, which are known to also cross-talk with one another through downstream cascades, may be important central pathways that explain the interconnection between the prognostic biomarkers identified. These results may suggest that these signaling pathways may represent new host immunomodulatory targets that warrant future mechanistic studies. Follow-up studies in model organisms and controlled studies would aid in establishing whether the genes identified in this study drive susceptibility, and in uncovering further mechanistic insights.

It is noteworthy that when comparing the current biomarker panels in the blunt trauma setting with that from our previous study among burn patients (Yan et al., 2015), none of the transcripts in the panels were shared. These differences may indicate that increased risk depends on the interaction of the type of trauma with each patient's underlying susceptibility to MIIE.

The present study describes methods towards the development of precision medicine tools and offers the possibility of analyses also for outcomes other than multiple infections. The failure of drug trials targeting sepsis (Marshall, 2014; Mitka, 2011) highlights the importance of further studies elucidating the underlying molecular mechanisms and components of heterogeneity in susceptibility to infection and infection-related morbidity within a population. Measuring the biomarker panels described herein to triage patients according to susceptibility to multiple infections can be used to strategically guide prophylactic patient management and help reduce the incidence of infections to limit sepsis (Boomer et al., 2011; Chaussabel, 2015; Parikh et al., 2016).

This study provides for the first time, prediction models for hypersusceptibility to infections, which is highly relevant for critically injured trauma patients, using a machine learning approach. A concern in general for prediction model building is that models may overfit to a specific dataset, making them less generalizable. However, using the multi-biomarker panel derived from the Glue Grant population to make predictions in the Cabrera et al. population yielded a relatively high AUROC, demonstrating the broader applicability of the biomarker panel model. These two populations had comparable injury severity; however, they were considerably different in their geographic locations and healthcare systems. Moreover, the gene expression levels were measured by two different transcriptome technologies (Illumina and Affymetrix), and the Cabrera et al. dataset was very small in sample size. Despite these differences, our multi-biomarker panels still conferred prediction, providing additional assurance in the validity of our results and evidence for the generalizability of our model. The present methods provide novel approaches for predicting outcomes from blood transcriptome information at admissions.

The value of early MIIE identification, prior to any clinical sign of infection, could be an indispensable tool in other types of trauma and to a wide range of clinical settings. Uncovering biomarkers of increased susceptibility to infections may open new avenues for novel therapeutic targets, as well as contribute to standardizing populations in clinical trials. Although predictive algorithms cannot eliminate medical uncertainty, our analysis method is expected to be widely applicable to other susceptible populations, such as those with diabetes or cardiac disease, the frail elderly population, those treated with immunosuppressive medication, as well as others. The described methodology of multi-biomarker panel development has the potential to be applied to outcomes and clinical contexts other than MIIE and trauma, providing additional value.

### Methods for Predicting Multiple Independent Infection Episodes (MIIE)

Provided herein are methods for detecting or predicting risk of developing multiple independent infection episodes (MIIE), i.e., two or more infections, in a subject who has experienced blunt trauma, but who does not presently have an infection. In some embodiments, the subject is a mammal, e.g., a human. The methods can be used to determine that the subject has, or has an increased risk of developing (i.e., a risk above that of a reference cohort of subjects with blunt trauma), an infection, e.g., developing two or more infections before discharge, e.g., within the next 2, 3, 4, 5, 6, 7, 10, 12, 14, 20, 21, or 24 days or more, e.g., within the next 3-120 days.

The infections can be, e.g., an infection with a Gram positive or Gram negative bacterium, virus, or fungus, e.g., Streptococcus pneumonia, Streptococcus viridans, Staphylococcus aureus, Enterococcus species, Coagulase negative staphylococci, and Streptococcus pneumoniae and viridian, Clostridium sp.; Candida species, Enterobacter species, Acinetobacter species, Pseudomonas aeruginosa, Haemophilus influenza, Bacteroides species, Klebsiella pneumoniae, Neisseria, Proteus, Serratia marcescens, Escherichia coli, Stenotrophomonas, and Candida species.

The methods include evaluating levels of biomarkers, e.g., the 15 probe set biomarker transcript panel or the 26 probe set biomarker transcript panel, in a sample. Preferably, the sample includes blood, serum, or plasma, e.g., comprising leukocytes, from a subject who has experienced blunt trauma, e.g., within the previous 72, 60, or 48 hours. The methods can include evaluating levels of subsets of the biomarker, e.g., 2, 3, 4, 5, 6, 7, 8, 9, 10, 11, 12, 13, 14, or all 15 transcripts in the 15 probe set biomarker panel.

In some embodiments, the methods include detecting one or more, e.g., 2, 3, 4, 5, 6, 7, 8, 9, 10, 11, 12, 13, 14, or all 15 transcripts in a 15 probe set biomarker panel comprising or consisting of hepatocyte growth factor (HGF); kelch repeat and BTB domain containing 7 (KBTBD7); adenosine A3 receptor (ADORA3); ADP-ribosylation factor-like GTPase 4A (ARL4A); epiplakin 1 (EPPK1); zinc finger protein 354A (ZNF354A); SH3 and PX domains 2B (SH3PXD2B); RNase A family 1 (RNASE1), BTB domain containing 19 (BTBD19), zeta chain of T cell receptor associated protein kinase 70 kDa (ZAP70); endoplasmic reticulum aminopeptidase 2 (ERAP2); CD96 molecule (CD96); membrane metallo-endipeptidase (MME); killer cell lectin-like receptor subfamily F, member 1 (KLRF1); and/or non-coding transcript, nuclear paraspeckle assembly transcript 1 (NEAT1) using a method known in the art.

In some embodiments, the methods include determining a level of all of ZNF354A; EPPK1; RNASE1; BTBD19; ADORA3; KBTBD7; SH3PXD2B; CD96; ARL4A; ZAP70; ERAP2; HGF; KLRF1; NEAT1; and MME.

In some embodiments, the methods also include determining a level of one or more of Interleukin 1 receptor, type II (IL1R2) and mannose receptor, C type 1 (MRC1); importin 11 (IPO11); dedicator of cytokinesis 4 (DOCK4); Kruppel-like factor 9 (KLF9); nebulette (NEBL); a different probe set for MME; ribosomal protein S6 kinase, 90 kDa, polypeptide 5 (RPS6KA5); killer cell lectin-like receptor, subfamily K, member 1 (KLRK1); granzyme K (GZMK); and/or ADP-ribosylation factor-like GTPase 4C (ARL4C).

In some embodiments, the methods include determining a level of one or more of Interleukin 1 receptor, type II (IL1R2) and mannose receptor, C type 1 (MRC1); importin 11 (IPO11); dedicator of cytokinesis 4 (DOCK4); Kruppel-like factor 9 (KLF9); nebulette (NEBL); a different probe set for MME; ribosomal protein S6 kinase, 90 kDa, polypeptide 5 (RPS6KA5); killer cell lectin-like receptor, subfamily K, member 1 (KLRK1); granzyme K (GZMK); and/or ADP-ribosylation factor-like GTPase 4C (ARL4C).

In some embodiments, the methods include determining a level of ARL4C; KLRK1; SH3PXD2B; EPPK1; ZNF354A; RNASE1; MME; BTBD19; MRC1; ADORA3; NEBL; KBTBD7; IPO11; RPS6KA5; KLF9; DOCK4; IL1R2; CD96; ARL4A; ERAP2; GZMK; NEAT1; KLRF1; HGF; MME; and ZAP70.

In some embodiments, the methods can include isolating nucleic acids from the samples. Various methods are well known within the art for the identification and/or isolation and/or purification of a biological marker from a sample. An “isolated” or “purified” biological marker is substantially free of cellular material or other contaminants from the cell or tissue source from which the biological marker is derived, i.e., partially or completely altered or removed from the natural state through human intervention. For example, nucleic acids contained in the sample can be first isolated according to standard methods, for example using lytic enzymes, chemical solutions, or isolated by nucleic acid-binding resins following the manufacturer's instructions.

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; RNA sequencing (RNA-Seq); Northern blot; various types of nucleic acid sequencing (Sanger, pyrosequencing, Next Generation Sequencing (NGS)); fluorescent in-situ hybridization (FISH); or hybridization-based approaches such as microarrays/chips, e.g., performed by commercially available equipment following manufacturer's protocols, e.g., using the Affymetrix GeneChip technology (Affymetrix, Santa 20 Clara, Calif.), Agilent (Agilent Technologies, Inc., Santa Clara, Calif.), or Illumina (Illumina, Inc., San Diego, Calif.) microarray technology) or Digital multiplexed gene expression analysis using the NanoString nCounter system (Kulkarni et al., Curr Protoc Mol Biol. 2011 April; Chapter 25:Unit25B.10), or barcoding methods (e.g., Serial Analysis of Gene Expression (SAGE)) (see also Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., gene chips or microarrays as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Hardiman, Microarrays Methods and Applications: Nuts & Bolts, DNA Press, 2003), can be used to detect the presence and/or level of the biomarkers. Measurement of the level of a biomarker can be direct or indirect. For example, the abundance levels of the biomarkers can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such as Beta-actin, GAPDH and Beta-tubulin, can be used.

In some embodiments, blood transcriptome microarrays can be used. Arrays can be prepared by selecting probes that hybridize to a polynucleotide sequence of a target biomarker, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be, e.g., synthesized either enzymatically in vivo, enzymatically in vitro (e.g. by PCR), or non-enzymatically in vitro.

In some embodiments, the probe sets used in the present methods comprises or consists of probes for the biomarkers listed herein, as well as housekeeping or other genes for normalization. In some embodiments, the methods include determining levels of transcripts in addition to the biomarkers described herein, but the methods include calculating a score based on only the biomarkers described herein.

In some embodiments, the presence and/or level of a biomarker is comparable to the presence and/or level of the biomarker(s) in the disease reference, then the subject has an increased risk of developing MIIE

In some embodiments, an increase in one, two, three, four, or more of, e.g., 14 or 15 of: hepatocyte growth factor (HGF); kelch repeat and BTB domain containing 7 (KBTBD7); adenosine A3 receptor (ADORA3); ADP-ribosylation factor-like GTPase 4A (ARL4A); epiplakin 1 (EPPK1); zinc finger protein 354A (ZNF354A); SH3 and PX domains 2B (SH3PXD2B); RNase A family 1 (RNASE1), or BTB domain containing 19 (BTBD19), or a decrease in one or more of: zeta chain of T cell receptor associated protein kinase 70 kDa (ZAP70); endoplasmic reticulum aminopeptidase 2 (ERAP2); CD96 molecule (CD96); membrane metallo-endipeptidase (MME); killer cell lectin-like receptor subfamily F, member 1 (KLRF1); and/or non-coding transcript, nuclear paraspeckle assembly transcript 1 (NEAT1), indicates that the subject has, or has an increased risk of developing (i.e., a risk above that of a reference cohort of subjects with blunt trauma), an infection, e.g., within the next 2, 3, 4, 5, 6, 7, 10, 12, 14, 20, 21, or 24 days. In some embodiments, SH3PXD2B is not evaluated.

In some embodiments, an increase in one or more of interleukin 1 receptor, type II (IL1R2); mannose receptor, C type 1 (MRC1); importin 11 (IPO11); dedicator of cytokinesis 4 (DOCK4); Kruppel-like factor 9 (KLF9); and/or nebulette (NEBL), or a decrease in MME, ribosomal protein S6 kinase, 90 kDa, polypeptide 5 (RPS6KA5); killer cell lectin-like receptor, subfamily K, member 1 (KLRK1); granzyme K (GZMK); and/or ADP-ribosylation factor-like GTPase 4C (ARL4C) indicates that the subject has, or has an increased risk of developing (i.e., a risk above that of a reference cohort of subjects with blunt trauma), an infection, e.g., within the next 2, 3, 4, 5, 6, 7, 10, 12, 14, 20, 21, or 24 days.

In some embodiments, the methods include calculating a score based on presence and/or level of a biomarker as described herein, and the score is comparable to a disease reference score (e.g., is above or below a threshold score), then the subject has an increased risk of developing MIIE In some embodiments, the levels of the biomarkers are used to calculate a score. The score can be calculated, e.g., using an algorithm such as summation, or weighted summation, of the (normalized) levels of the biomarkers. Specific algorithms can be identified using known statistical methods including PCA, linear regression, SVM (support vector machine), decision tree, KNN (K-nearest neighbors), K-means, gradient boosting, or random forest methods.

Thus, in some embodiments, to assess whether a subject has an increased risk of developing MIIE, the method can include first log transforming the biomarker values and then assigning a predicted probability, e.g., using a logistic regression model, to produce a probability score. If a subject has a predicted probability score above a selected threshold, e.g., at least 50%, 60%, 70%, 80%, or 90%, the subject would be predicted to have an increased risk of developing MIIE If the predicted probability score is below the selected threshold, e.g., 50%, the subject would be predicted to have a normal risk of developing MIIE

For example, in some embodiments, an exemplary model uses a logistic regression analysis to calculate a probability score, wherein each variable (biomarker, X) gets a weight (B). In the exemplary equation below, the weights (B) are calculated for each marker, and there can be unique B values for each of the biomarkers, e.g., for each of the biomarkers.

$$L n ( \frac{P}{1 - P} ) = {β}_{0} + {β}_{1} {X}_{1} + {β}_{2} {X}_{2} + … + {β}_{k} {X}_{k}$$

The coefficients of the model provide a weighting of the predictors to produce the predicted response variable value. In some embodiments, e.g., for the 15 marker panel, the coefficients are:

| Predictors | Coefficient Estimates |
|---|---|
| Intercept | 1.67158 |
| BTBD19 | 0.52495 |
| RNASE1 | 0.57518 |
| MME | −0.6925 |
| ARL4A | −0.10097 |
| ZNF354A | 0.78963 |
| CD96 | −0.06368 |
| HGF | −0.43305 |
| ZAP70 | −0.20691 |
| KLRF1 | −0.48816 |
| ADORA3 | 0.47734 |
| ERAP2 | −0.40628 |
| KBTBD7 | 0.35635 |
| SH3PXD2B | 0.14436 |
| EPPK1 | 0.66524 |
| NEAT1 | −0.63188 |

Using these exemplary coefficients, algorithms for calculating patients' predicted probability of multiple infections based on levels of each of the biomarkers are:

Logit(MIIE)=1.67158+0.52495BTBD19+0.57518RNASE1 −0.6925MME−0.10097ARL4A+0.78963ZNF354A−0.06368CD96−0.43305HGF−0.20691ZAP70 −0.48816KLRF1+0.47734ADORA3−0.40628ERAP2+0.35635KBTBD7+0.14436SH3PXD2B+0.66524EPPK1 −0.63188NEAT1

And

Odds(MIIE)=e\hat{ }(1.67158+0.52495BTBD19+0.57518RNASE1 −0.6925MME−0.10097ARL4A+0.78963ZNF354A−0.06368CD96—0.43305HGF−0.20691ZAP70 −0.48816KLRF1+0.47734ADORA3−0.40628ERAP2+0.35635KBTBD7+0.14436SH3PXD2B+0.66524EPPK1 −0.63188NEAT1)

And finally:

$$Probability ( MIIE ) = \frac{Odds ( MIIE )}{1 + Odds ( MIE )}$$

In some embodiments, additional variables are considered, e.g., presence of comorbidities including diabetes or cardiovascular disease, age, or injury severity scores, e.g., APACHEII, ISS, NISS.

In some embodiments, the amount by which the level (or score) in the subject is less than the reference level (or score) is sufficient to distinguish a subject from a control subject, and optionally is a statistically significantly less than the level (or score) in a control subject. In cases where the level (or score) of the biomarker(s) in a subject being equal to the reference level (or score) of the biomarker(s), the “being equal” refers to being approximately equal (e.g., not statistically different).

Suitable reference values can be determined using methods known in the art, e.g., using standard clinical trial methodology and statistical analysis. The reference values can have any relevant form. In some cases, the reference comprises a predetermined value for a meaningful score or level of a biomarker, e.g., a control reference level that represents a normal level of a biomarker, e.g., a level in an unaffected subject or a subject who is not at risk of developing MIIE, and/or a disease reference that represents a level of the proteins associated with risk of developing MIIE

The predetermined level or score can be a single cut-off (threshold) value, such as a median or mean, or a level or score that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level or score is a level or score determined in the same subject, e.g., at a different time point, e.g., an earlier time point.

Subjects associated with predetermined values are typically referred to as reference subjects. For example, in some embodiments, a control reference subject is one who has a blunt trauma and later experiences 0 or 1 infection events.

A disease reference subject is one who has a blunt trauma and who later experiences 2 or more infections (MIIE).

An increased risk is defined as a risk above the risk of subjects in the general population of subjects who have blunt trauma.

The predetermined value can depend upon the particular population of subjects (e.g., human subjects) selected. For example, an apparently healthy population will have a different ‘normal’ range of levels or scores than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

Methods of Treatment

In some embodiments, once it has been determined that a person has an increased risk of developing MIIE, then a treatment, e.g., as known in the art or as described herein, can be administered.

The present methods can be used to facilitate clinical decision-making by effectively discriminating between those who are likely to develop multiple infections and those who are not. In some embodiments, patients who are found not to be hypersusceptible to MIIE (i.e., to have a lower risk of developing MIIE) can continue to receive the currently established standard of care. In some embodiments, the methods include treating subjects identified as hypersusceptible to or at risk of developing MIIE, e.g., by administering a broad-spectrum antibiotic, increasing the frequency or length of monitoring of the subject for infection; implementing prophylactic measures; and/or enhancing patient nutrition; those who are identified to be at high risk could can be treated with from increased surveillance and additional preventative measures taken early. Some antibiotics include vancomycin; linezolid; daptomycin; telavancin; doxycycline; minocycline; aminoglycosides; ampicillin; amoxicillin/clavulanic acid (augmentin); azithromycin; carbapenems (e.g. imipenem, doripenem); piperacillin/tazobactam; quinolones (e.g. ciprofloxacin); tetracycline-class drugs; tigecycline; chloramphenicol; ticarcillin; colistin and tigecycline combination therapy; and trimethoprim/sulfamethoxazole (bactrim); see, e.g., Pakyz, “Broad-spectrum antibiotics,” Infectious Disease Advisor, 2017 (available at infectiousdiseaseadvisor.com/home/decision-support-in-medicine/hospital-infection-control/broad-spectrum-antibiotics/). Additional interventions for the high risk group can include increased surveillance for early mobilization and removal of lines/tubes, coating IV lines and urine catheters with antimicrobials and/or antibiotics, immunomodulatory nutrition therapies (Aghaeepour et al., 2017; Lorenz et al., 2015), and microbiome alterations (Harris et al., 2017; Tosh and McDonald, 2012).

Such additional measures would incur unnecessary costs if implemented in all trauma patients, however, could be cost-effective when used in this targeted set of patients. Efforts aimed at increased prevention have the potential to contribute to alleviating the current antibiotic resistance crisis, toxicity of antibiotics, and the imposed burden on healthcare costs. Accurate outcome prediction and risk stratification methodologies, such as one we describe here, could be valuable amid crisis situations that result in severe hospital overload with critically ill patients and scarcity of medical resources. The ability to identify patients at low risk for specific morbidity and mortality can be used as an aid to informed prioritization of resource allocation to patients with better potential for recovery and survival (Massachusetts, 2020; Medicine, 2013; Medicine, 2020).

### EXAMPLES

The invention is further described in the following examples, which do not limit the scope of the invention described in the claims.

Materials and Methods

The following materials and methods were used in the Examples herein.

Study design/patients

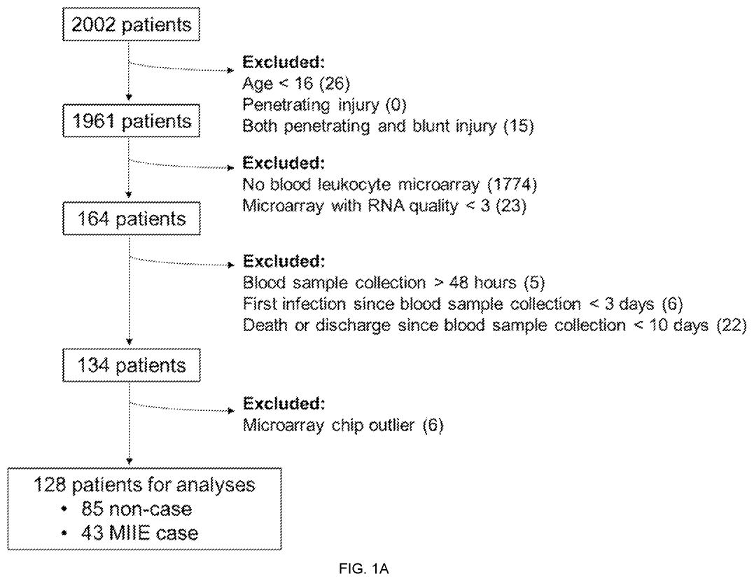

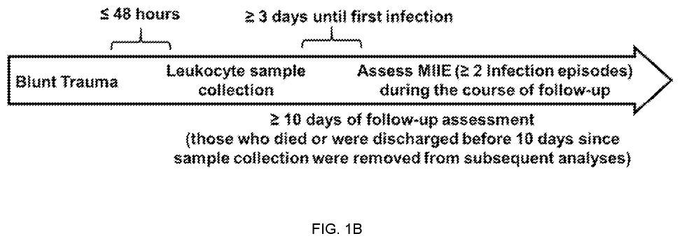

We performed a secondary analysis of patient clinical and genomic data from the Glue Grant, a prospective, longitudinal study that enrolled patients at seven US Level 1 trauma centers between 2003 and 2009. Permission for the use of de-identified data was obtained from the Massachusetts General Hospital Institutional Review Board. Among the 2,002 patients in the dataset, inclusion criteria identified 128 adult (age ≥16 years) patients who suffered blunt trauma (excluding penetrating injury only, or blunt with penetrating injury), had follow-up time of at least 10 days since blood collection, and had early (≤48 hours since trauma injury) blood microarray transcriptome data of high RNA quality (RNA quality ≥3 out of 4) and chips that were not outliers. At least three days from time of blood draw to first recorded infection (FIG. 1A, B). Where patients had multiple blood samples that fit the criteria, the earliest was used. Clinical outcomes and injury severity scores (APACHEII, ISS, NISS, Denver 2, Marshall) were recorded by participating institutions according to the guidelines outlined by the Glue Grant Consortium. Body mass index (BMI) was calculated from recorded height and weight, and BMI categories were assigned according to the Centers for Disease Control and Prevention's recommendations. For assigning categories of major clinical procedures, recorded sub-categories were consolidated to create more general categories. Similarly, various non-infection complications and surgical site infection sub-categories were consolidated, as indicated below.

Major Clinical Procedures Categories:

For assigning categories of major clinical procedures, sub-categories were combined as follows: “Laparotomy” includes entries of laparotomy NOS, and laparotomy with other procedure, with splenectomy, with repair/packing of the liver, with the repair of gastric, duodenal or small bowel perforation, with the repair of the large bowel or rectal perforation, with nephrectomy, with repair of major vascular injury, with drainage of intra-abdominal abscess, and second-look laparotomy or abdominal washout. “Orthopedic” includes soft tissue debridement/amputations, internal fixation of femur, open skeletal fixation exclusive of femur, percutaneous skeletal fixation, and spine fixation. “Vascular” includes peripheral vascular and angiographic embolization. “Craniotomy” and “Thoracotomy” were used as recorded in the original dataset.

Surgical Site Infections Categories:

For surgical site infection categories, “Superficial incisional infection” includes recordings of the shoulder girdle, lumbar spine (bony), lower extremity, and abdomen and pelvis (non-bony) indicated as superficial incisional and “Deep incisional” combines upper extremity, lower extremity, pelvis (body), and abdomen and pelvis (non-body) recorded as deep incisional, as well as chest (pleural space), abdomen and pelvis (non-bony), upper extremity, and lower extremity recorded as organ/space.

Outcome Definition:

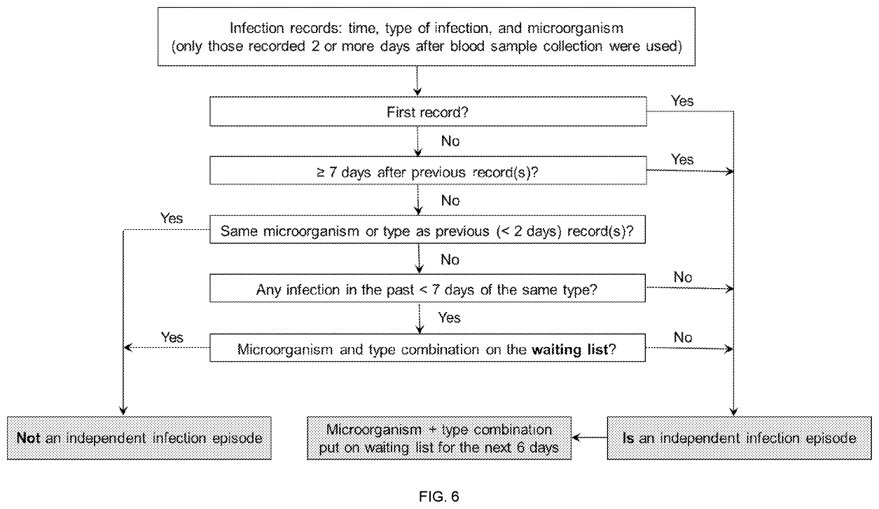

Patients were assigned to MBE-cases (≥2 cumulative infection episodes) and non-cases (≤1 infection episode), following the same method as in our previous study (Yan et al., 2015), in which a decision tree considering the timing and type of infection and the isolated pathogen was used to tabulate total independent infection episodes (FIG. 6).

Quantification and Statistical Analysis

Software Used

R version 3.4.4 was used for the statistical analyses, as described below, with the following packages and versions: GCRMA 2.50.0 (Wu and Irizarry, 2017), arrayQualityMetrics 3.34.0 (Kauffmann et al., 2009), EMA 1.4.5 (Servant et al., 2010), LIMMA 3.34.9 (Ritchie et al., 2015), Glmnet 2.0-16 (Friedman et al., 2010), pROC 1.13.0 (Robin et al., 2011), Caret 6.0-81 (Kuhn, 2008), epiR 0.9-99 (Stevenson et al., 2019).

Statistical Analyses

Baseline characteristics are reported as medians with interquartile range, means with standard deviation, or total numbers with proportions in percentages, as indicated in the legend (Table 1A). Medians between two groups (non-cases versus MIIE-cases) were compared using the Mann-Whitney U test, and means were analyzed by the unpaired t-test assuming equal variance. For comparing proportions, the Chi-square test was used for expected values 5 or greater, or Fisher's exact test was used for an expected value less than 5.

For processing microarray data files, first, the GCRMA package was employed to obtain normalized log 2 expression values of probe sets. Then chips that were flagged as outliers by the arrayQualityMetrics package were excluded. Subsequently, internal control probe sets were removed, and the EMA package was used to filter out low abundance probe sets (below the threshold of 3.5 log 2 expression value across all samples). These filtering steps reduced the number of probe sets from 54,675 to 25,567 for subsequent analyses. The LIMMA package was used to identify probe sets with at least 1.5-fold difference between the non-cases and MIIE-cases; this reduced the number of probe sets to 137. Functional annotation analyses were conducted using Database for Annotation, Visualization, and Integrated Discovery (DAVID), version 6.8 (Huang da et al., 2009), using the 1.5-fold changed 137 probe sets as the target and total 25,567 probe sets as the background set. The fold enrichment, unadjusted and FDR-adjusted p-values were reported.

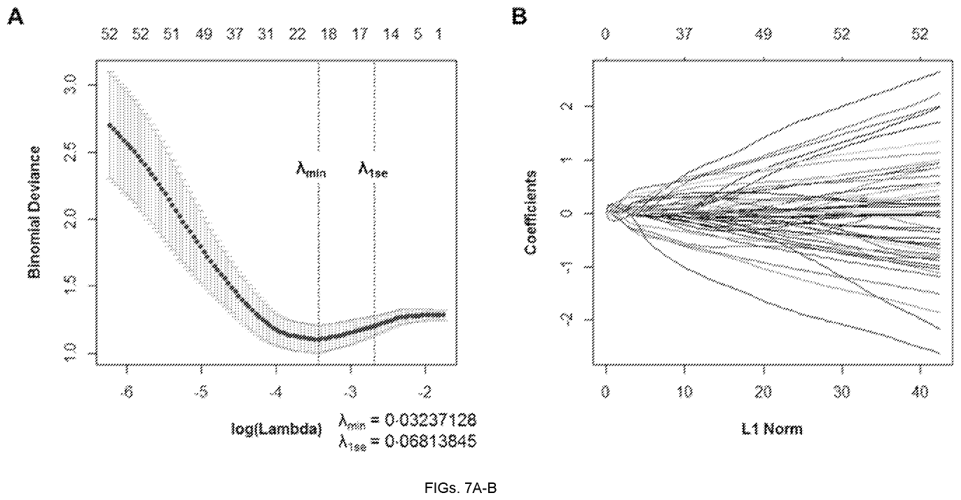

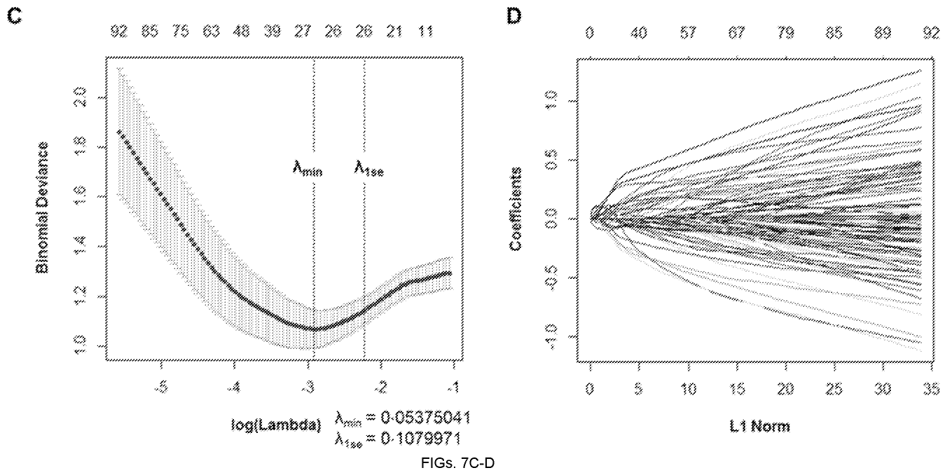

The Glmnet package was used to implement the Least absolute shrinkage and selection operator (LASSO) regression and Elastic Net regression, to select probe sets that were predictive of MIIE, as previously conducted (Yan et al., 2015). The penalty weight, lambda (k), was identified by finding the 10-fold cross-validation (CV) error, repeated 100 times. Probe sets were selected according to the value of k that yielded minimum average binomial deviance plus one standard error on the test set (λ1se), rather than minimum (λmin) to limit overfitting. For LASSO, the λ1se was found to be 0.068, and for the Elastic net, it was found to be 0.108 (FIG. 7). This biomarker panel selection process yielded 15 probe sets with LASSO and 26 probe sets with Elastic net. For building the clinical model, similarly to our previous study (Yan et al., 2015), stepwise regression was conducted to select each of the severity scores (APACHEII, ISS, NISS), with potential other covariates. For APACHEII, which includes age as one of the factors for the score calculation, sex and race were used for stepwise selection. ISS and NISS were each tested with age, sex, and race. For all the severity scores, univariate models with the scores only were selected.

The following logistic regression models were constructed for the binary MIIE outcome: (a) multivariate model with the 15 probe set LASSO predictors, (b) multivariate model with the 26 probe sets Elastic Net predictors, (c) univariate models of various clinical scores (APACHEII ISS, NISS), and (d) multivariate models with a combination of the 15, or 26 probe sets with each of the clinical scores. The predictors were identified with LASSO or Elastic Net. Then, a logistic regression model with those 15 (LASSO) or 26 (Elastic Net) predictors was fit by maximum likelihood. The area under the receiver operating characteristic curve (AUROC) with bootstrap 95% confidence intervals were estimated and used to compare the models using the pROC package. The comparison was repeated for 10-fold cross-validation sampling using the Caret package. Sensitivity, specificity, positive predictive value (PPV), and negative predictive value (NPV) and confidence intervals were calculated using the epiR package, using the optimal probability cut-off determined as the top-left corner of the ROC curve for each model.

For the volcano plot, log 2 fold change comparing MIIE-cases and non-cases was plotted on the x-axis and p-values on the y-axis, of all initial 25,567 probe sets after the filtering step. Data points of probe sets with at least 1.5-fold expression level difference (137 probe sets) are marked with black dots. Among these 137 probe sets, those corresponding to the 15 probe set panel selected with LASSO are marked with orange squares, and additional probe sets selected by Elastic Net for the 26 probe set panel are marked with blue triangles. Ingenuity Pathway Analyses (IPA) was used to generate an interaction network of the 26 probe sets selected by Elastic Net (QIAGEN) (Kramer et al., 2014).

External Validation Dataset and Statistical Analyses

Blood transcriptome data for external validation was obtained from the Cabrera et al., which conducted a secondary analysis of the Activation of Coagulation and Inflammation in Trauma (ACIT2) cohort (Cabrera et al., 2017). The ACIT2 study enrolled adult trauma patients at the Royal London Hospital, and the transcriptome study was conducted among patients enrolled between 2008-2012. The datasets with normalized Illumina microarray log 2 expression levels, accompanying clinical information, and infections outcome were downloaded from: github.com/C4TS/HyperacutePhase. For our analysis, we included 28 unique critically injured trauma patients with baseline ISS and infections outcome information available, after removing those with missing information. The Cabrera et al. data set does not have the resolution of the infection outcome that our Glue grant data set does; therefore we classified infection “yes” or “no” status, as indicated in their dataset. This subpopulation's ISS range was 25-51 (median 31), and the age range was 17-69 (median 37.5), which were comparable to the Glue Grant population. Among them, 26 had blood transcriptome data available for the 24 hour time point, and 25 had data available for the 72 hour time. Expression values of all the genes represented in the 15 probe set panel, except SH3PXD2B, was available in the Cabrera et al. dataset (“NormData” file). The data processing steps in Cabrera et al. entailed filtering out “nonexpressed” probe sets that did not pass the detection p-value threshold of 0.05 in at least three arrays. Therefore, we assumed the expression values for SH3PXD2B to be 0 for our analysis. We applied the 15 probe set panel model to calculate the predicted probabilities for the subjects in the Cabrera et al. dataset and constructed the ROC curve.

### Example 1. Patient Demographics and Baseline Characteristics

Baseline characteristics of the 128 blunt trauma patients included in the study (FIG. 1A, B) are presented in Table 1A. Motor vehicle collisions were the most frequent injury mechanisms (Table 1B). The overall study population consisted of approximately 61.7% males and 38.3% females, with median age [IQR] of 34 [25-44] years (Table 1A). Common demographic factors were not significantly different between non-cases (≤1 infection episode) and MIIE-cases (≥2 infection episodes). However, baseline injury severity scores were significantly higher for MIIE-cases (APACHEII score 27 [24-31] vs. 29 [26-33.5], p=0.008; ISS 29 [18-41] vs. 36 [27-41.5], p=0.041; NISS 34 [27-43] vs. 41 [30-50], p=0.045, respectively), while AIS (highest score in any body region) was comparable.

As expected, orthopedic procedures were the most frequent surgical interventions that patients received overall (76.6%), followed by laparotomy (47.7%), vascular procedures (23.4%), thoracotomy (6.3%) and craniotomy (1.6%) (Table 1A). Apart from the proportion of patients having undergone laparotomy, which was significantly higher among MIIE-cases compared to non-cases (41.2% vs. 60.5%, p=0.04), other procedures were similar between non-cases and MIIE-cases.

There were five total patients who did not survive, and the cause of death was different for each (Table 1C). Mortality was similar between non-cases (3.5%) and MIIE-cases (4.7%, p=1.00). Among survivors, MIIE-cases had significantly longer hospital stay than non-cases (discharge at day 19 [14-26.75] vs. 35 [27-47], p<0.0001). MIIE-cases also had a higher proportion of those experiencing non-infection complications compared to non-cases (47.1% vs. 81.4%, p=0.0002). Maximum Denver and Marshall scores were significantly higher for MIIE-cases compared to non-cases (Denver score 2 [0-3] vs. 3 [2-3.5], p<0.0001; Marshall score 4.7 [3.3-6.4] vs. 6.9 [5.8-8.1], p<0.0001). Among the sub-categories that together determined the total Marshall Score, maximum scores of the following were significantly higher for MIIE-cases compared to non-cases: cardio (2.4 [1.7-3.2] vs. 2.8 [2.8-4.0], p<0.0001), respiratory (2.4 [1.7-3.2] vs. 2.8 [2.1-3.1], p<0.0001), and hepatic score (0.0 [0.0-0.8] vs. 0.7 [0.0-1.5], p<0.001). The maximum central nervous system, renal and hematologic scores were not significantly different.

TABLE 1A
Baseline characteristics, for the outcome of multiple
independent infection episodes (MIIE)

| | Non-case, ≤1 | MIIE case, ≥2 | |
|---|---|---|---|
| | infection | infection | |
| All patients | episode | episodes | |
| (N = 128) | (n = 85) | (n = 43) | p-value |

DEMOGRAPHIC INFORMATION/CLINICAL DESCRIPTORS

| | | | | | | | |
|---|---|---|---|---|---|---|---|
| Age | 34 | [25-44] | 35 | [26-44] | 32 | [23.5-46] | 0.718^{a} |

Sex

| | | | | | | | |
|---|---|---|---|---|---|---|---|
| Female | 49 | (38.3%) | 30 | (35.3%) | 19 | (44.1%) | 0.328^{c} |
| Male | 79 | (61.7%) | 58 | (64.7%) | 23 | (55.8%) | |

| | | | | |
|---|---|---|---|---|
| BMI | 29.3 ± 7.0 | 28.8 ± 7.0 | 30.1 ± 6.9 | 0.325^{b} |

BMI categories

| | | | | | | | |
|---|---|---|---|---|---|---|---|
| Underweight | 1 | (0.8%) | 0 | (0%) | 1 | (2.3%) | 0.336^{d} |
| Healthy | 37 | (28.9%) | 28 | (32.9%) | 9 | (20.9%) | 0.216^{d} |
| Overweight | 39 | (30.5%) | 26 | (30.6%) | 13 | (27.9%) | 1.000^{d} |
| Obese | 51 | (39.8%) | 31 | (36.5%) | 20 | (46.5%) | 0.340^{d} |

INJURY CHARACTERISTICS

| | | | | | | | |
|---|---|---|---|---|---|---|---|
| Crush injury | 19 | (14.8%) | 16 | (18.8%) | 3 | (7.0%) | 0.113^{d} |
| Severe head injury | 13 | (10.2%) | 8 | (9.4%) | 5 | (11.6%) | 0.760^{d} |

Severity Scores

| | | | | | | | |
|---|---|---|---|---|---|---|---|
| APACHE II | 28 | [24-32] | 27 | [24-31] | 29 | [27-33.5] | 0.008^{a} |
| ISS | 34 | [22-41.25] | 29 | [18-41] | 36 | [27-41.5] | 0.041^{a} |
| NISS | 37 | [27-43.5] | 34 | [27-43] | 41 | [30-50] | 0.045^{a} |
| AIS (highest of any | 4 | [3-5] | 4 | [3-5] | 4 | [3.5-5] | 0.587^{a} |
| body part) | | | | | | | |

CLINICAL Events
Post-trauma interventions

| | | | | | | | |
|---|---|---|---|---|---|---|---|
| Craniotomy | 2 | (1.6%) | 1 | (1.2%) | 1 | (2.3%) | 0.402^{d} |
| Thoracotomy | 8 | (6.3%) | 5 | (5.9%) | 3 | (7.0%) | 1.000^{d} |
| Laparotomy | 61 | (47.7%) | 35 | (41.2%) | 26 | (60.5%) | 0.042^{d} |
| Orthopedic | 98 | (76.6%) | 61 | (71.8%) | 37 | (86.0%) | 0.081^{d} |
| Vascular | 30 | (23.4%) | 19 | (22.4%) | 11 | (25.6%) | 0.667^{d} |

CLINICAL OUTCOMES

| | | | | | | | |
|---|---|---|---|---|---|---|---|
| Mortality | 5 | (3.9%) | 3 | (3.5%) | 2 | (4.7%) | 1.000^{d} |
| Days in ICU | 11 | [6-18] * | 8 | [5-14.75] * | 20 | [15-29] * | <0.0001^{a} |
| | 11.5 | [6-18.25] | 8 | [5-15] | 20 | [14.5-28] | <0.0001^{a} |
| Discharge day since | 23 | [16.5-33] * | 19 | [14-26.75] * | 35 | [27-47] * | <0.0001^{a} |
| injury | 23 | [16-33] | 19 | [13-26] | 34 | [25.5-47] | <0.0001^{a} |
| Days on ventilator in | 8 | [4-15] | 6 | [3-11] | 15 | [10-23] | <0.0001^{a} |
| ICU | | | | | | | |
| Non-infection | 75 | (58.6%) | 40 | (47.1%) | 35 | (81.4%) | 0.0002^{c} |
| complications | | | | | | | |
| Maximum Denver 2 | 2 | [1-3] | 2 | [0-3] | 3 | [2-3.5] | <0.0001^{a} |
| score | | | | | | | |
| Maximum Marshall | 5.8 | [4.0-7.2] | 4.7 | [3.3-6.4] | 6.9 | [5.8-8.1] | <0.0001^{a} |
| score | | | | | | | |
| Maximum central | 4 | [4-4] | 4 | [4-4] | 4 | [4-4] | 0.064^{a} |
| nervous system | | | | | | | |
| score | | | | | | | |
| Maximum cardio | 2.8 | [2.0-3.6] | 2.4 | [1.7-3.2] | 3.3 | [2.8-4.0] | <0.0001^{a} |
| score | | | | | | | |
| Maximum | 2.8 | [2.0-3.6] | 2.4 | [1.7-3.2] | 2.8 | [2.1-3.1] | <0.0001^{a} |
| respiratory score | | | | | | | |
| Maximum renal | 0.8 | [0.7-1.0] | 0.8 | [0.7-0.9] | 0.9 | [0.7-1.4] | 0.063^{a} |
| score | | | | | | | |
| Maximum hepatic | 0.0 | [0.0-1.2] | 0.0 | [0.0-0.8] | 0.7 | [0.0-1.5] | 0.002^{a} |
| score | | | | | | | |
| Maximum | 0.5 | [0.0-1.1] | 0.5 | [0.0-1.0] | 0.6 | [0.2-1.1] | 0.224^{a} |
| hematologic score | | | | | | | |

Median [q1-q3], or mean ± SD for continuous variables and n (%) for categorical variables are reported. P-values calculations are indicated as: aMann-Whitney U two-tailed test, bunpaired equal variance two-tailed Student's t-test, cChi-square, dFisher's Exact two-tailed test.
*ICU days and discharge day since injury when calculated only among survivors.

TABLE 1B
Injury mechanisms among all 128 patients
in the study, related to TABLE 1A.
Injury mechanisms

| | | | | | | | |
|---|---|---|---|---|---|---|---|
| Fall | 7 | (5.5%) | 6 | (7.1%) | 1 | (2.3%) | 0.42^{d} |
| Machinery | 1 | (0.8%) | 1 | (1.2%) | 0 | (0%) | 1.00^{d} |
| MVC - occupant | 70 | (54.7%) | 43 | (50.6%) | 27 | (62.8%) | 0.26^{d} |
| MVC - motorcyclist | 23 | (18.0%) | 16 | (18.8%) | 7 | (16.3%) | 0.81^{d} |
| MVC - cyclist | 3 | (2.3%) | 3 | (3.5%) | 0 | (0%) | 0.55^{d} |
| MVC - pedestrian | 19 | (14.8%) | 13 | (15.3%) | 6 | (14.0%) | 1.00^{d} |
| Assault | 1 | (0.8%) | 1 | (1.2%) | 0 | (0%) | 1.00^{d} |
| Other | 4 | (3.1%) | 2 | (2.4%) | 2 | (4.7%) | 0.60^{d} |

TABLE 1C
Causes of death among the 5 non-survivors
in the study, related to Table 1A.

| Death day | Primary cause | Secondary cause | |
|---|---|---|---|
| since injury | of death | of death | MIIE case |
| 10 | Multiple | | No |
| | organ failure | | |
| 10 | Sepsis | Multiple | No |
| | | organ failure | |
| 20 | Hypoxia | | No |
| 11 | Severe head | Cardiac | Yes |
| | injury trauma | dysfunction | |
| 24 | Brain death | | Yes |

### Example 2. Characteristics of Infection Episodes

(i) Patient Case Outcomes and Timing of Infection Onset

Among the 128 patients in the study, there were 85 non-cases—42 with no infection and 43 with one infection episode—and 42 were MIIE-cases (i.e., ≥2 infection episodes). The median [IQR] day for detection of first infection episode, among those who experienced at least one infection episode (i.e., excluding 42 patients those who had no infection episode, for a total of 86 patients) was 8 [5-12] days (Table 2).

(ii) Incidence of Surgical Site Infections Versus Other Nosocomial Infections

Among all 128 patients, 36 (28.1%) experienced surgical site infections, compared to 80 (62.5%) who experienced other nosocomial infections (Table 2). Comparing specific subtypes of nosocomial infections, pneumonia (39.1% overall) was highest, followed by urinary tract infection (18.8%), blood infection (17.2%), pseudomembranous colitis (3.9%), catheter-related bloodstream infection (3.9%), empyema (2.3%), and other unspecified infections (5.5%).

(iii) Microorganism Detection

When comparing the incidence of various microorganisms among non-cases with one infection episode versus MIIE-cases, relatively higher proportion was found for MIIE-cases specifically for Gram positives of Staphylococcus aureus (18.2% vs. 39.5%), Enterococcus species (11.4% vs. 25.6%), Coagulase negative staphylococci (2.3% vs. 16.3%), and Streptococcus pneumoniae and viridans (2.3% vs. 4.7% for both). The incidence of the Gram positive, Clostridium species was the same for non-cases and MIIE-cases (both 7.0%) (Table 2). For Gram negative bacteria, the incidences of the following microorganisms were higher for MIIE-cases compared to non-cases: Enterobacter species (11.4% vs. 37.2%), Acinetobacter species (9.1% vs. 30.2%), Pseudomonas aeruginosa (11.4% vs. 16.3%), Haemophilus influenza (4.5% vs. 14.0%), Bacteroides species (0% vs. 9.3%), Klebsiella pneumoniae (2.3% vs. 7.0%), Neisseria (0% vs. 7.0%), Proteus (2.3% vs. 4.7%), Serratia marcescens (2.3% vs. 4.7%), and Gram negative, not otherwise specified (NOS) (2.3% vs. 11.6%). The incidence was higher among non-cases than MIIE-cases for Escherichia coli (11.4% vs. 4.7%), and Stenotrophomonas (4.6% vs. 0%). Fungi incidences were higher among MIIE-cases compared to non-cases: Candida species (9.1% vs. 11.6%) and unspecified fungi (0% vs. 2.3%).

(iv) Timing of Microorganism Detection

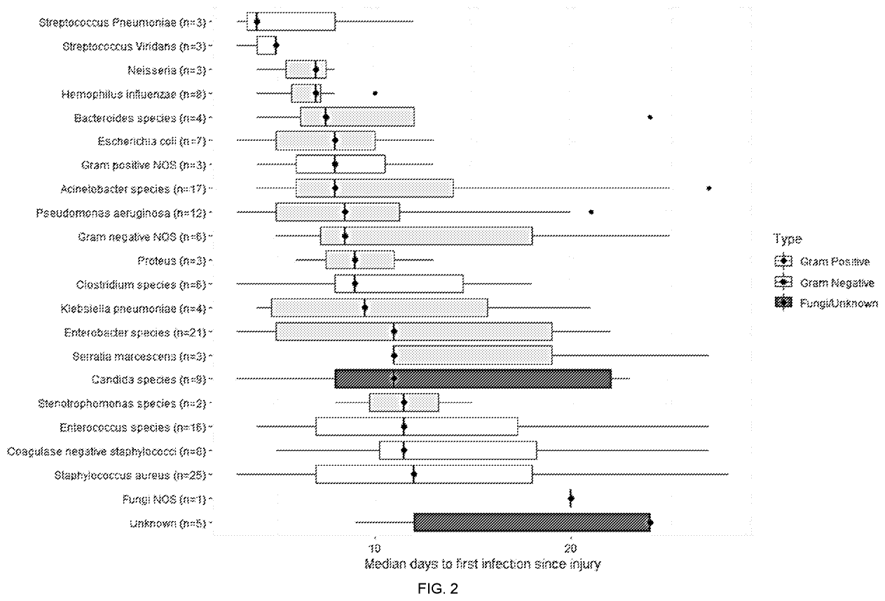

The median time to the first day of detection of different microorganisms ranged widely (FIG. 2). The Gram positives, Streptococcus pneumoniae, and Streptococcus viridans were found first (at median day 4 and 5, respectively), followed by various Gram negatives and the Gram positive, Clostridium species, between median day 7 and 9.5. Microorganisms that appeared relatively later (day 11 to 12) included Candida species, Enterobacter species, and Serratia marcescens, Stenotrophomonas, Enterococcus, Coagulase negative staphylococci and Staphylococcus aureus.

TABLE 2
Patient Outcomes.

| | 0 and 1 | | |
|---|---|---|---|
| | infection | 1 infection | ≥2 infection |
| All | episode | episode only | episodes |
| patients | (non-cases) | (non-cases) | (MIIE case) |
| (N = 128) | (n = 85) | (n = 43) | (n = 43) |

CHARACTERISTICS OF INFECTION

| | | | | | | | | |
|---|---|---|---|---|---|---|---|---|
| First infection day since | 8 | [5-12] * | 8 | [5.5-12] * | 8 | [5.5-12] | 7 | [4-11.5] |
| injury | | | | | | | | |
| Surgical Site Infections | 36 | (28.1%) | 7 | (8.2%) | 7 | (16.3%) | 29 | (67.4%) |
| Superficial incisional | 7 | (5.5%) | 2 | (2.4%) | 2 | (4.7%) | 5 | (11.6%) |
| Deep incisional | 30 | (23.4%) | 5 | (5.9%) | 5 | (11.6%) | 25 | (62.8%) |
| Other infection sites | 80 | (62.5%) | 37 | (43.5%) | 37 | (86.0%) | 43 | (100%) |
| Pneumonia | 50 | (39.1%) | 20 | (23.5%) | 20 | (46.5%) | 30 | (69.8%) |
| Ventilation-associated | 46 | (35.9%) | 20 | (23.5%) | 20 | (46.5%) | 26 | (60.5%) |
| pneumonia | | | | | | | | |
| Bloodstream infection | 22 | (17.2%) | 4 | (4.7%) | 4 | (9.3%) | 18 | (41.9%) |
| Urinary tract infection | 24 | (18.8%) | 7 | (8.2%) | 7 | (16.3%) | 17 | (39.5%) |
| Pseudomembranous colitis | 5 | (3.9%) | 3 | (3.5%) | 3 | (7.0%) | 2 | (4.7%) |
| Catheter-related | 5 | (3.9%) | 1 | (1.2%) | 1 | (2.3%) | 4 | (9.3%) |
| bloodstream infection | | | | | | | | |
| Empyema | 3 | (2.3%) | 2 | (2.4%) | 2 | (4.7%) | 1 | (2.3%) |
| Other | 7 | (5.5%) | 2 | (2.4%) | 2 | (4.7%) | 5 | (11.6%) |

Organism incidence

| | | | | | | | | |
|---|---|---|---|---|---|---|---|---|
| Gram positive bacteria | 51 | (39.8%) | 20 | (23.5%) | 20 | (46.5%) | 31 | (72.1%) |
| Staphylococcus aureus | 25 | (19.5%) | 8 | (9.4%) | 8 | (18.2%) | 17 | (39.5%) |
| Enterococcus species | 16 | (12.5%) | 5 | (5.9%) | 5 | (11.4%) | 11 | (25.6%) |
| Coagulase negative | 8 | (6.3%) | 1 | (1.2%) | 1 | (2.3%) | 7 | (16.3%) |
| staphylococci | | | | | | | | |
| Clostridium species | 6 | (4.7%) | 3 | (3.5%) | 3 | (7.0%) | 3 | (7.0%) |
| Streptococcus | 3 | (2.3%) | 1 | (1.2%) | 1 | (2.3%) | 2 | (4.7%) |
| pneumoniae | | | | | | | | |
| Streptococcus viridans | 3 | (2.3%) | 1 | (1.2%) | 1 | (2.3%) | 2 | (4.7%) |
| Gram positive NOS | 3 | (2.3%) | 1 | (1.2%) | 1 | (2.3%) | 2 | (4.7%) |
| Gram negative bacteria | 57 | (44.5%) | 23 | (27.1%) | 23 | (53.5%) | 34 | (79.1%) |
| Enterobacter species | 21 | (16.4%) | 5 | (5.9%) | 5 | (11.4%) | 16 | (37.2%) |
| Acinetobacter species | 17 | (13.3%) | 4 | (4.7%) | 4 | (9.1%) | 13 | (30.2%) |
| Pseudomonas aeruginosa | 12 | (9.4%) | 5 | (5.9%) | 5 | (11.4%) | 7 | (16.3) |
| Haemophilus influenza | 8 | (6.3%) | 2 | (2.4%) | 2 | (4.5%) | 6 | (14.0%) |
| Escherichia coli | 7 | (5.5%) | 5 | (5.9%) | 5 | (11.4%) | 2 | (4.7%) |
| Bacteroides species | 4 | (3.1%) | 0 | (0%) | 0 | (0%) | 4 | (9.3%) |
| Klebsiella pneumoniae | 4 | (3.1%) | 1 | (1.2%) | 1 | (2.3%) | 3 | (7.0%) |
| Neisseria | 3 | (2.3%) | 0 | (0%) | 0 | (0%) | 3 | (7.0%) |
| Proteus | 3 | (2.3%) | 1 | (1.2%) | 1 | (2.3%) | 2 | (4.7%) |
| Serratia marcescens | 3 | (2.3%) | 1 | (1.2%) | 1 | (2.3%) | 2 | (4.7%) |
| Stenotrophomonas | 2 | (1.6%) | 2 | (2.4%) | 2 | (4.6%) | 0 | (0%) |
| species | | | | | | | | |
| Gram negative NOS | 6 | (4.7%) | 1 | (1.2%) | 1 | (2.3%) | 5 | (11.6%) |
| Fungi | 10 | (7.8%) | 4 | (4.7%) | 4 | (9.3%) | 6 | (14.0%) |
| Candida species | 9 | (7.0%) | 4 | (4.7%) | 4 | (9.1%) | 5 | (11.6%) |
| Fungi NOS | 1 | (0.8%) | 0 | (0%) | 0 | (0%) | 1 | (2.3%) |
| Unknown | 5 | (3.9%) | 2 | (2.4%) | 2 | (2.3%) | 3 | (7.0%) |

Numbers represent individuals who have experienced the indicated outcomes.
*The 42 non-cases who had 0 infection episode, without recordings for first infection day were omitted for these calculations. Recorded species are indicated or described as not otherwise specified (NOS).

### Example 3. Gene Ontology (GO) Reports of 1.5 Fold Changed Probe Sets

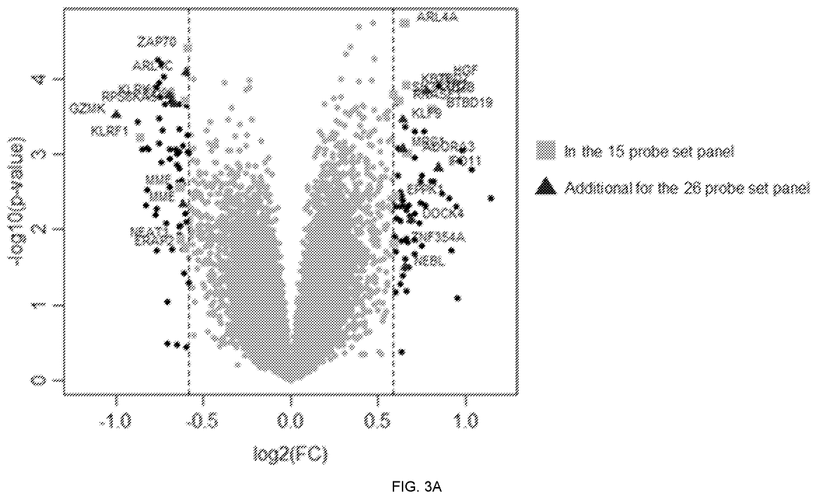

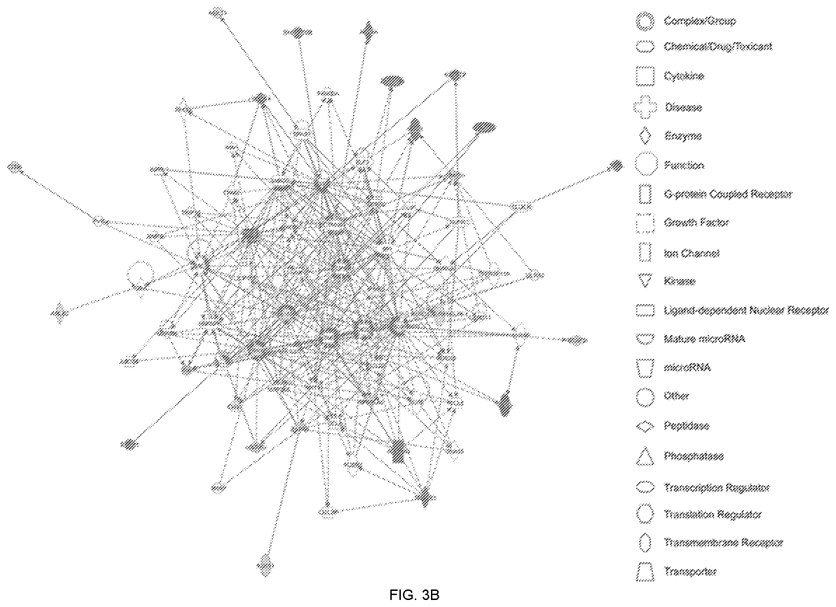

We identified 137 probe sets showing at least 1.5-fold up- or down-regulated difference in expression levels between non-cases and MIIE-cases (FIG. 3A), and performed Gene Ontology (GO) analyses to find enriched biological processes and KEGG pathway terms. As expect, terms relevant to various immune response pathways were among the top fold enrichment. They included interleukin-4 secretion, regulation of interferon-gamma secretion, cytolysis, regulation of natural killer cell-mediated cytotoxicity, viral genome replication, cellular defense response, adaptive immune response, humoral immune response, T cell co-stimulation, regulation of immune response, T cell receptor signaling pathway, response to tumor necrosis factor, response to virus, immune response and innate immune response. Other biological processes terms with high fold enrichment were signaling pathways with relevance to cell proliferation and differentiation, including regulation of p38 MAPK kinase, calcium-mediated signaling, regulation of fat cell differentiation, organ regeneration, MAP kinase activity, and phosphatidylinositol 3-kinase (PI3K) signaling. Similarly, KEGG pathway terms with high fold enrichment included those relevant to immune response, including natural killer cell-mediated cytotoxicity, malaria, hematopoietic cell lineage, and T cell receptor signaling pathway. Additionally, regulation of cancer, which may have overlapping signaling pathways with infections-related terms, and related to tissue regeneration, also appeared among the enriched terms. Although according to the FDR-adjusted p-values, statistical significance in enrichment was detected only for a small number of terms, the fold enrichment ranking consistently points to the relevance of immune response terms, as expected.

### Example 4. Prognostic Biomarker Panel Selection

To identify a multi-biomarker panel that collectively predicts the outcome of MIIE, we analyzed the 137 differentially-regulated probe sets by employing a machine learning pipeline that we previously developed and successfully used in our previous study among burn patients (Yan et al., 2015). In the current study, we further added to our analyses pipeline by utilizing a combination of LASSO and Elastic Net regression methods. The LASSO regression that reduces redundancy in predictor selection would allow for a narrow selection of a minimal biomarker panel, which is expected to be more practical. Elastic Net regression that includes correlated predictors allowed for a more comprehensive discovery of additional probe sets that are potentially biologically relevant. With LASSO, 15 probe sets were selected, mostly relevant to immune functions and signaling cascades for cellular proliferation and differentiation (Table 3; FIG. 3A; FIGS. 7A, B). Upregulated probe sets in the minimal 15 probe set panel included those associated with immune function terms, hepatocyte growth factor (HGF) and ketch repeat and BTB domain containing 7 (KBTBD7); signaling cascades, adenosine A3 receptor (ADORA3), ADP-ribosylation factor-like GTPase 4A (ARL4A), epiplakin 1 (EPPK1), zinc finger protein 354A (ZNF354A), SH3 and PX domains 2B (SH3PXD2B); and those with no function term assigned, RNase A family 1 (RNASE1), and BTB domain containing 19 (BTBD19). Downregulated probe sets included those with immune functions, zeta chain of T cell receptor associated protein kinase 70 kDa (ZAP70), endoplasmic reticulum aminopeptidase 2 (ERAP2), CD96 molecule (CD96), membrane metallo-endipeptidase (WE), and killer cell lectin-like receptor subfamily F, member 1 (KLRF1); and a non-coding transcript, nuclear paraspeckle assembly transcript 1 (NEAT1) (Table 3; FIG. 3A).

With Elastic Net, a total of 26 probe sets were selected that included the 15 probe sets from LASSO and 11 additional ones (Table 3; FIG. 3A; FIGS. 7C, D). The additional upregulated probe sets consisted of those with immune functions, interleukin 1 receptor, type II (IL1R2) and mannose receptor, C type 1 (MRC1); those involved with signaling including, importin 11 (IPO11), dedicator of cytokinesis 4 (DOCK4) and Kruppel-like factor 9 (KLF9); as well as nebulette (NEBL) that is important for muscle filament assembly. The additional downregulated probe sets included a different probe set for WE, and other immune function genes, ribosomal protein S6 kinase, 90 kDa, polypeptide 5 (RPS6KA5), killer cell lectin-like receptor, subfamily K, member 1 (KLRK1) and granzyme K (GZMK); and the signaling molecule, ADP-ribosylation factor-like GTPase 4C (ARL4C) (Table 3; FIG. 3A).

TABLE 3
Probe Sets

| | | | | In both 15 and |
|---|---|---|---|---|
| | | | Fold | 26 probe set |
| | | | Change | panel, or |
| | Gene | | (MIIE/non- | uniquely in 26 |
| Probe set | Symbol | Gene Name | cases) | probe set panel |
| 210998_s_at | HGF | hepatocyte growth factor | 1.85 | 15 and 26 |
| 238488_at | IPO11 | importin 11 | 1.80 | 26 |
| 1557049_at | BTBD19 | BTB (POZ) domain containing 19 | 1.76 | 15 and 26 |
| 211372_s_at | IL1R2 | interleukin 1 receptor, type II | 1.72 | 26 |
| 205003_at | DOCK4 | dedicator of cytokinesis 4 | 1.61 | 26 |
| 223660_at | ADORA3 | adenosine A3 receptor | 1.59 | 15 and 26 |
| 229970_at | KBTBD7 | kelch repeat and BTB (POZ) | 1.59 | 15 and 26 |
| | | domain containing 7 | | |
| 203962_s_at | NEBL | nebulette | 1.58 | 26 |
| 205020_s_at | ARL4A | ADP-ribosylation factor like | 1.57 | 15 and 26 |
| | | GTPase 4A | | |
| 203543_s_at | KLF9 | Kruppel-like factor 9 | 1.56 | 26 |
| 204438_at | MRC1 | mannose receptor, C type 1 | 1.56 | 26 |
| 201785_at | RNASE1 | ribonuclease, RNase A family 1 | 1.54 | 15 and 26 |
| 232164_s_at | EPPK1 | epiplakin 1 | 1.52 | 15 and 26 |
| 205427_at | ZNF354A | zinc finger protein 354A | 1.51 | 15 and 26 |
| 231823_s_at | SH3PXD2B | SH3 and PX domains 2B | 1.50 | 15 and 26 |
| 214032_at | ZAP70 | zeta chain of T cell receptor- | 0.67 | 15 and 26 |
| | | associated protein kinase 70 kDa | | |
| 202208_s_at | ARL4C | ADP-ribosylation factor-like | 0.66 | 26 |
| | | GTPase 4C | | |
| 227462_at | ERAP2 | endoplasmic reticulum | 0.66 | 15 and 26 |
| | | aminopeptidase 2 | | |
| 206761_at | CD96 | CD96 molecule | 0.66 | 15 and 26 |
| 203434_s_at | MME | membrane metallo-endopeptidase | 0.65 | 26 |
| 238320_at | NEAT1 | nuclear paraspeckle assembly | 0.65 | 15 and 26 |
| | | transcript 1 (non-protein coding) | | |
| 203435_s_at | MME | membrane metallo-endopeptidase | 0.65 | 15 and 26 |
| 204633_s_at | RPS6KA5 | ribosomal protein S6 kinase, | 0.63 | 26 |
| | | 90 kDa, polypeptide 5 | | |
| 1555691_a_at | KLRK1 | killer cell lectin-like receptor | 0.62 | 26 |
| | | subfamily K, member 1 | | |
| 220646_s_at | KLRF1 | killer cell lectin-like receptor | 0.55 | 15 and 26 |
| | | subfamily F, member 1 | | |
| 206666_at | GZMK | granzyme K | 0.50 | 26 |

Fold Change and p-values of the 15 probe set panel from LASSO and 26 probe set panel from elastic net, in order of magnitude of up- (values above 1) to down- (values less than 1) regulated.

### Example 5. Pathway Network Suggests the Relevance of Major Pathways

We assessed the molecular network connection among the 26 probe sets that were selected by Elastic Net (FIG. 3B). The major nodes with the most extensive edges that were central to the connections consisted of major signaling pathway components that are key regulators of inflammation, mitogentic response, and tissue regeneration. These notable nodes included tumor necrosis factor (TNF), transforming growth factor beta-1 (TGFβ-1), and various chemokine (C—X—C motif) ligand (CXCL) members and chemokine (C—C motif) ligand (CCL) members. TNF and TGFβ-1 are major cytokines that can act both synergistically or antagonistically to each other, depending on the cell context. Further substantiating the involvement of these factors, we found p38 mitogen-activated protein kinase (MAPK), which can act downstream of both TNF-α and TGFβ-1 pathways. The other major nodes, extracellular signal-regulated kinases 1/2 (ERK1/2), and mothers against decapentaplegic homolog 3 (SMAD3) are also key downstream components of the TGFβ pathway. A key downstream transcriptional regulator for the canonical Wnt pathway, β-catenin (CTNNB1), another major pathway known to cross-talk with TGFβ, was also found as a major node in this network.

### Example 6. Performance in Prediction of MIIE Using the Multi-Biomarker Panel Versus the Clinical Severity Models

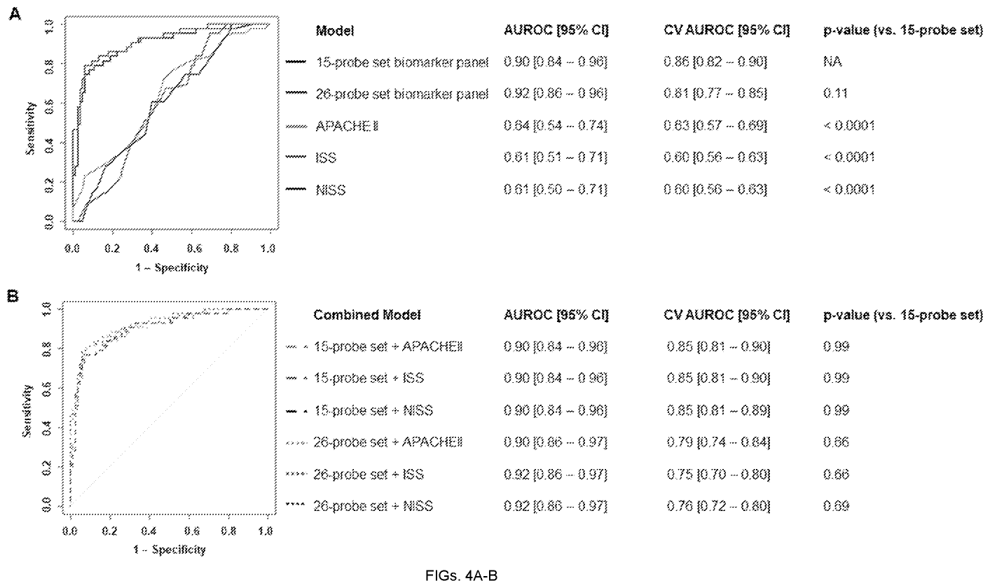

The AUROC [95% CI] of the logistic regression model for predicting MBE outcome with the 15 probe set biomarker panel developed with LASSO was 0.90 [0.84-0.96] (FIG. 4A). For the 26 probe set panel developed with Elastic Net, there was marginal AUROC increase (0.92 [0.86-0.96], p=0.11) (FIG. 4A). Given the objective of the study of developing measures taken early upon arrival at the hospitals for predicting future outcome during recovery, we evaluated common injury severity scores. Compared to the biomarker models, the AUROC of the various injury severity models was notably lower (0.64 [0.54-0.74] for APACHEII, 0.611 [0.51-0.71] for ISS, and 0.61 [0.50-0.71] for NISS, all p<0.0001 compared to the 15 probe set panel model) (FIG. 4A). The Odds Ratios (OR) [95% CI] for all the covariates of each of the models were also found (Tables 4, 5, 6).

The 15 and 26 probe set panel models had sensitivity [95% CI] of 0.74 [0.59-0.86] vs. 0.79 [0.64-0.90] respectively; specificity [95% CI] of 0.94 [0.87-0.98] vs. 0.94 [0.87-0.98]; positive predictive value (PPV) of 0.86 [0.71-0.95] vs. 0.87 [0.73-0.96]; and negative predictive value (NPV) of 0.88 [0.79-0.94] vs. 0.90 [0.82-0.95] (Table 7). For the various injury severity scores (APACHEII, ISS, NISS), the sensitivity, specificity, PPV, and NPV were generally lower compared to the multi-biomarker panel models (Table 7).

Moreover, we constructed multivariate logistic regression models combining the 15, or the 26 probe set panel with each of the clinical injury severity scores. The AUROC [95% CI] of the 15 probe set panel combined with APACHEII was 0.90 [0.84-0.96], with ISS it was 0.902 [0.84-0.96], and with NISS it was 0.902 [0.84-0.96] (FIG. 4B). For the 26 probe set panel, the combination with APACHEII was 0.90 [0.86-0.97]; with ISS, it was 0.92 [0.86-0.97], and with NISS it was 0.92 [0.86-0.97] (FIG. 4B). None of the combined models were significantly different from their respective biomarker only models, suggesting that the addition of clinical score information does not improve the prediction.

TABLE 4
OR estimates [95% CI] and coefficient p-values of the clinical
severity score univariate models, related to FIG. 4

| Clinical models | OR [95% CI] | p-value |
|---|---|---|
| APACHE II | 1.11 [1.03-1.19] | <0.01** |
| ISS | 1.03 [1.00-1.06] | 0.06 |
| NISS | 1.03 [1.00-1.06] | 0.06 |

TABLE 5
OR estimates [95% CI] and coefficient p-values of the
biomarker panel multivariate models, related to FIG. 4.

| 15 probe set biomarker panel multivariate model: | | | 26 probe set biomarker panel multivariate model: | | | |
|---|---|---|---|---|---|---|
| MODEL | OR [95% CI] | p-value | MODEL | OR [95% CI] | | p-value |
| 205427_at: | 2.20 [1.42-3.81] | <0.01** | 202208_s_at: | 4.12 | [0.47-44.23] | 0.21 |
| ZNF354A | | | ARL4C | | | |
| 232164_s_at: | 1.94 [1.14-3.50] | 0.02* | 1555691_a_at: | 3.70 | [0.97-17.46] | 0.07 |
| EPPK1 | | | KLRK1 | | | |
| 201785_at: | 1.78 [0.90-3.82] | 0.11 | 231823_s_at: | 2.58 | [0.65-13.11] | 0.21 |
| RNASE1 | | | SH3PXD2B | | | |
| 1557049_at: | 1.69 [0.82-3.69] | 0.17 | 232164_s_at: | 2.30 | [1.21-4.86] | 0.02* |
| BTBD19 | | | EPPK1 | | | |
| 223660_at: | 1.61 [0.81-3.48] | 0.20 | 205427_at: | 2.27 | [1.30-4.45] | <0.01** |
| ADORA3 | | | ZNF354A | | | |
| 229970_at: | 1.43 [0.65-3.26] | 0.38 | 201785_at: | 2.12 | [0.92-5.56] | 0.09 |
| KBTBD7 | | | RNASE1 | | | |
| 231823_s_at: | 1.16 [0.45-3.17] | 0.77 | 203434_s_at: | 2.10 | [0.22-25.75] | 0.54 |
| SH3PXD2B | | | MME | | | |
| 206761_at: | 0.94 [0.23-3.55] | 0.93 | 1557049_at: | 1.97 | [0.76-5.25] | 0.16 |
| CD96 | | | BTBD19 | | | |
| 205020_s_at: | 0.90 [0.28-2.80] | 0.86 | 204438_at: | 1.92 | [0.77-5.29] | 0.18 |
| ARL4A | | | MRC1 | | | |
| 214032_at: | 0.81 [0.22-3.13] | 0.76 | 223660_at: | 1.76 | [0.75-4.59] | 0.21 |
| ZAP70 | | | ADORA3 | | | |
| 227462_at: | 0.67 [0.41-1.03] | 0.08 | 203962_s_at: | 1.45 | [0.94-2.29] | 0.10 |
| ERAP2 | | | NEBL | | | |
| 210998_s_at: | 0.65 [0.29-1.35] | 0.26 | 229970_at: | 1.43 | [0.42-5.17] | 0.57 |
| HGF | | | KBTBD7 | | | |
| 220646_s_at: | 0.61 [0.34-1.06] | 0.09 | 238488_at: | 1.24 | [0.58-2.76] | 0.58 |
| KLRF1 | | | IPO11 | | | |
| 238320_at: | 0.53 [0.32-0.83] | <0.01** | 204633_s_at: | 1.17 | [0.36-3.93] | 0.79 |
| NEAT1 | | | RPS6KA5 | | | |
| 203435_s_at: | 0.50 [0.25-0.91] | 0.04* | 203543_s_at: | 1.01 | [0.29-3.57] | 0.99 |
| MME | | | KLF9 | | | |
| | | | 205003_at: | 0.96 | [0.55-1.75] | 0.90 |
| | | | DOCK4 | | | |
| | | | 211372_s_at: | 0.85 | [0.20-3.55] | 0.83 |
| | | | IL1R2 | | | |
| | | | 206761_at: | 0.80 | [0.12-5.05] | 0.81 |
| | | | CD96 | | | |
| | | | 205020_s_at: | 0.60 | [0.14-2.30] | 0.47 |
| | | | ARL4A | | | |
| | | | 227462_at: | 0.58 | [0.32-0.97] | <0.05* |
| | | | ERAP2 | | | |
| | | | 206666_at: | 0.57 | [0.21-1.48] | 0.26 |
| | | | GZMK | | | |
| | | | 238320_at: | 0.39 | [0.18-0.73] | <0.01** |
| | | | NEAT1 | | | |
| | | | 220646_s_at: | 0.35 | [0.14-0.82] | 0.02* |
| | | | KLRF1 | | | |
| | | | 210998_s_at: | 0.33 | [0.09-0.99] | 0.07 |
| | | | HGF | | | |
| | | | 203435_s_at: | 0.31 | [0.03-2.38] | 0.28 |
| | | | MME | | | |
| | | | 214032_at: | 0.28 | [0.03-2.04] | 0.22 |
| | | | ZAP70 | | | |

TABLE 6
OR estimates [95% CI] and coefficient p-values of the combined genomic(15 or
26 probe set panel) and clinical severity score multivariate models, related to FIG. 4.

| | +APACHE II | | | +ISS | | | +NISS | | |
|---|---|---|---|---|---|---|---|---|---|
| MODEL | OR [95% CI] | | p-value | OR [95% CI] | | p-value | OR [95% CI] | | p-value |
| 202208_s_at: | 3.89 | [0.45-41.33] | 0.23 | 3.74 | [0.40-45.88] | 0.27 | 4.32 | [0.45-54.91] | 0.23 |
| ARL4C | | | | | | | | | |
| 1555691_a_at: | 3.76 | [0.99-17.66] | 0.07 | 3.66 | [0.96-17.26] | 0.07 | 3.74 | [0.97-17.79] | 0.07 |
| KLRK1 | | | | | | | | | |
| 231823_s_at: | 2.64 | [0.65-13.84] | 0.21 | 2.50 | [0.63-12.95] | 0.23 | 2.62 | [0.65-13.77] | 0.21 |
| SH3PXD2B | | | | | | | | | |
| 232164_s_at: | 2.30 | [1.20-4.84] | 0.02* | 2.26 | [1.18-4.85] | 0.02* | 2.32 | [1.21-4.95] | 0.02* |
| EPPK1 | | | | | | | | | |
| 201785_at: | 2.20 | [0.94-5.73] | 0.08 | 2.10 | [0.92-5.50] | 0.10 | 2.28 | [1.30-4.50] | <0.01** |
| RNASE1 | | | | | | | | | |
| 205427_at: | 2.18 | [1.22-4.34] | 0.01* | 2.26 | [1.29-4.42] | <0.01** | 2.13 | [0.92-5.69] | 0.09 |
| ZNF354A | | | | | | | | | |
| 1557049_at: | 1.98 | [0.75-5.41] | 0.17 | 1.91 | [0.72-5.24] | 0.20 | 2.11 | [0.22-25.96] | 0.53 |
| BTBD19 | | | | | | | | | |
| 204438_at: | 1.92 | [0.77-5.33] | 0.18 | 1.91 | [0.76-5.28] | 0.18 | 2.00 | [0.76-5.47] | 0.16 |
| MRC1 | | | | | | | | | |
| 203434_s_at: | 1.79 | [0.17-23.44] | 0.64 | 2.08 | [0.22-25.72] | 0.54 | 1.93 | [0.77-5.35] | 0.18 |
| MME | | | | | | | | | |
| 223660_at: | 1.77 | [0.75-4.72] | 0.21 | 1.71 | [0.71-4.56] | 0.25 | 1.78 | [0.74-4.78] | 0.21 |
| ADORA3 | | | | | | | | | |
| 203962_s_at: | 1.45 | [0.94-2.30] | 0.10 | 1.44 | [0.93-2.28] | 0.11 | 1.45 | [0.94-2.30] | 0.10 |
| NEBL | | | | | | | | | |
| 229970_at: | 1.44 | [0.43-5.18] | 0.56 | 1.44 | [0.43-5.24] | 0.56 | 1.42 | [0.42-5.16] | 0.57 |
| KBTBD7 | | | | | | | | | |
| 204633_s_at: | 1.26 | [0.37-4.41] | 0.71 | 1.18 | [0.36-3.96] | 0.78 | 1.24 | [0.57-2.76] | 0.59 |
| RPS6KA5 | | | | | | | | | |
| 238488_at: | 1.25 | [0.57-2.80] | 0.58 | 1.24 | [0.58-2.75] | 0.57 | 1.16 | [0.35-3.92] | 0.80 |
| IPO11 | | | | | | | | | |
| 203543_s_at: | 0.97 | [0.27-3.49] | 0.96 | 0.99 | [0.28-3.56] | 0.99 | 1.01 | [0.29-3.56] | 0.99 |
| KLF9 | | | | | | | | | |
| 205003_at: | 0.96 | [0.54-1.74] | 0.89 | 0.97 | [0.55-1.77] | 0.91 | 0.96 | [0.54-1.75] | 0.89 |
| DOCK4 | | | | | | | | | |
| 211372_s_at: | 0.88 | [0.20-3.75] | 0.86 | 0.87 | [0.20-3.65] | 0.85 | 0.85 | [0.20-3.54] | 0.82 |
| IL1R2 | | | | | | | | | |
| 206761_at: | 0.83 | [0.12-5.27] | 0.85 | 0.81 | [0.12-5.04] | 0.82 | 0.80 | [0.11-5.06] | 0.81 |
| CD96 | | | | | | | | | |
| 205020_s_at: | 0.64 | [0.14-2.64] | 0.54 | 0.61 | [0.14-2.39] | 0.49 | 0.59 | [0.13-2.31] | 0.46 |
| ARL4A | | | | | | | | | |
| 227462_at: | 0.59 | [0.32-0.99] | 0.06 | 0.57 | [0.31-0.96] | <0.05* | 0.58 | [0.32-0.97] | 0.05 |
| ERAP2 | | | | | | | | | |
| 206666_at: | 0.58 | [0.21-1.51] | 0.28 | 0.60 | [0.20-1.63] | 0.33 | 0.57 | [0.20-1.50] | 0.26 |
| GZMK | | | | | | | | | |
| 238320_at: | 0.39 | [0.19-0.75] | <0.01** | 0.39 | [0.19-0.74] | <0.01** | 0.38 | [0.18-0.74] | <0.01** |
| NEAT1 | | | | | | | | | |
| 220646_s_at: | 0.37 | [0.14-0.86] | 0.03* | 0.36 | [0.14-0.86] | 0.03* | 0.35 | [0.13-0.84] | 0.03* |
| KLRF1 | | | | | | | | | |
| 203435_s_at: | 0.37 | [0.03-3.23] | 0.39 | 0.31 | [0.03-2.47] | 0.29 | 0.33 | [0.08-1.01] | 0.08 |
| MME | | | | | | | | | |
| 210998_s_at: | 0.33 | [0.09-0.98] | 0.06 | 0.35 | [0.09-1.10] | 0.10 | 0.30 | [0.03-2.37] | 0.28 |
| HGF | | | | | | | | | |
| 214032_at: | 0.28 | [0.03-2.00] | 0.21 | 0.30 | [0.03-2.38] | 0.26 | 0.27 | [0.03-2.14] | 0.22 |
| ZAP70 | | | | | | | | | |
| Clinical Score | 1.02 | [0.92-1.14] | 0.69 | 1.01 | [0.95-1.06] | 0.80 | 1.00 | [0.94-1.05] | 0.90 |

TABLE 7
Sensitivity and specificity, PPV and NPV of the various models, related to FIG. 4.

| | | Corresponding | | | | | | | | |
|---|---|---|---|---|---|---|---|---|---|---|
| | Probability | clinical score | Sensitivity | | Specificity | | PPV | | NPV | |
| Model | threshold | cut-offs | [95% CI] | | [95% CI] | | [95% CI] | | [95% CI] | |
| 15 | 0.561 | NA | 0.74 | [0.59-0.86] | 0.94 | [0.87-0.98] | 0.86 | [0.71-0.95] | 0.88 | [0.79-0.94] |
| biomarker | | | | | | | | | | |
| panel | | | | | | | | | | |
| 26 | 0.476 | NA | 0.79 | [0.64-0.90] | 0.94 | [0.87-0.98] | 0.87 | [0.73-0.96] | 0.90 | [0.82-0.95] |
| biomarker | | | | | | | | | | |
| panel | | | | | | | | | | |
| APACHEII | 0.315 | 28.22 | 0.72 | [0.56-0.85] | 0.54 | [0.43-0.65] | 0.44 | [0.32-0.57] | 0.79 | [0.67-0.89] |
| ISS | 0.267 | 21.02 | 0.95 | [0.84-0.99] | 0.31 | [0.21-0.42] | 0.41 | [0.31-0.51] | 0.93 | [0.76-0.99] |
| NISS | 0.344 | 39.50 | 0.60 | [0.44-0.75] | 0.60 | [0.49-0.70] | 0.43 | [0.31-0.57] | 0.75 | [0.63-0.85] |
| 15 | 0.479 | NA | 0.77 | [0.61-0.88] | 0.93 | [0.85-0.97] | 0.85 | [0.69-0.94] | 0.89 | [0.80-0.94] |
| biomarker | | | | | | | | | | |
| panel + | | | | | | | | | | |
| APACHEII | | | | | | | | | | |
| 15 | 0.511 | NA | 0.77 | [0.61-0.88] | 0.93 | [0.85-0.97] | 0.85 | [0.69-0.94] | 0.89 | [0.80-0.94] |
| biomarker | | | | | | | | | | |
| panel + | | | | | | | | | | |
| ISS | | | | | | | | | | |
| 15 | 0.505 | NA | 0.77 | [0.61-0.88] | 0.93 | [0.85-0.97] | 0.85 | [0.69-0.94] | 0.89 | [0.80-0.94] |
| biomarker | | | | | | | | | | |
| panel + | | | | | | | | | | |
| NISS | | | | | | | | | | |
| 26 | 0.463 | NA | 0.81 | [0.67-0.92] | 0.92 | [0.84-0.97] | 0.83 | [0.69-0.93] | 0.91 | [0.82-0.96] |
| biomarker | | | | | | | | | | |
| panel + | | | | | | | | | | |
| APACHEII | | | | | | | | | | |
| 26 | 0.476 | NA | 0.79 | [0.64-0.90] | 0.94 | [0.87-0.98] | 0.87 | [0.73-0.96] | 0.90 | [0.82-0.95] |
| biomarker | | | | | | | | | | |
| panel + | | | | | | | | | | |
| ISS | | | | | | | | | | |
| 26 | 0.446 | NA | 0.81 | [0.67-0.92] | 0.92 | [0.84-0.97] | 0.83 | [0.69-0.93] | 0.91 | [0.82-0.96] |
| biomarker | | | | | | | | | | |
| panel + | | | | | | | | | | |
| NISS | | | | | | | | | | |

### Example 7. External Validation of the Multi-Biomarker Panel in a Different Cohort of Severe Trauma Patients

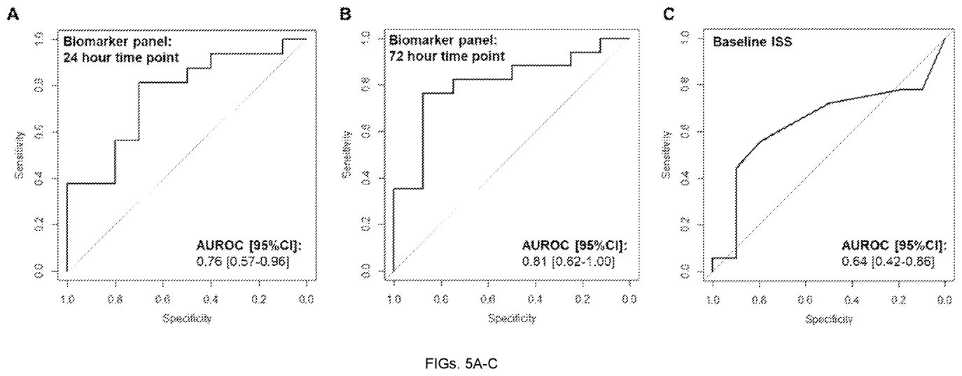

We performed external validation of our biomarker panel using a severe blunt trauma patient cohort from a previous publication by Cabrera et al. (Cabrera et al., 2017), which had post-admission blood transcriptome log₂ expression values available. The microarray platform used in this study was different from the one used in the Glue Grant, and the measure of one of the genes in the 15 probe set panel was missing. Despite the differences in measurement technology, the lack of measurement for one of the panel, and outcome resolution between the Glue Grant and the Cabrera et al. datasets (as described in detail in the methods section), the multi-biomarker panel achieved a relatively high AUROC of 0.76 [0.57-0.96] for the 24 hour post-admission dataset and 0.81 [0.62-1.00] for 72 hours post-admission (FIGS. 5A, B). On the other hand, the model with ISS (the only injury severity score that was shared with the Cabrera et al. dataset) had a much lower AUROC of 0.64 [0.42-0.86], which was not significantly above 0.5. These results provide evidence for the generalizability of the multi-biomarker panel described herein to different trauma cohorts.

### REFERENCES

Aghaeepour, N., Kin, C., Ganio, E. A., Jensen, K. P., Gaudilliere, D. K., Tingle, M., Tsai, A., Lancero, H. L., Choisy, B., McNeil, L. S., et al. (2017). Deep Immune Profiling of an Arginine-Enriched Nutritional Intervention in Patients Undergoing Surgery. J Immunol.
Baker, S. P., O'Neill, B., Haddon, W., Jr., and Long, W. B. (1974). The injury severity score: a method for describing patients with multiple injuries and evaluating emergency care. J Trauma 14, 187-196.
Barten, R., Torkar, M., Haude, A., Trowsdale, J., and Wilson, M. J. (2001). Divergent and convergent evolution of NK-cell receptors. Trends Immunol 22, 52-57.
Boomer, J. S., To, K., Chang, K. C., Takasu, O., Osborne, D. F., Walton, A. H., Bricker, T. L., Jarman, S. D., 2nd, Kreisel, D., Krupnick, A. S., et al. (2011).
Immunosuppression in patients who die of sepsis and multiple organ failure. JAMA 306, 2594-2605.
Cabrera, C. P., Manson, J., Shepherd, J. M., Torrance, H. D., Watson, D., Longhi, M. P., Hoti, M., Patel, M. B., O'Dwyer, M., Nourshargh, S., et al. (2017). Signatures of inflammation and impending multiple organ dysfunction in the hyperacute phase of trauma: A prospective cohort study. PLoS Med 14, e1002352.
Chaussabel, D. (2015). Assessment of immune status using blood transcriptomics and potential implications for global health. Semin Immunol 27, 58-66.
Cheadle, W. G., Wilson, M., Hershman, M. J., Bergamini, D., Richardson, J. D., and Polk, H. C., Jr. (1989). Comparison of trauma assessment scores and their use in prediction of infection and death. Ann Surg 209, 541-545; discussion 545-546.
Cole, E., Davenport, R., Willett, K., and Brohi, K. (2014). The burden of infection in severely injured trauma patients and the relationship with admission shock severity. J Trauma Acute Care Surg 76, 730-735.
Cuenca, A. G., Delano, M. J., Kelly-Scumpia, K. M., Moreno, C., Scumpia, P. O.,
Laface, D. M., Heyworth, P. G., Efron, P. A., and Moldawer, L. L. (2011). A paradoxical role for myeloid-derived suppressor cells in sepsis and trauma. Mol Med 17, 281-292.
Desai, K. H., Tan, C. S., Leek, J. T., Maier, R. V., Tompkins, R. G., Storey, J. D., Inflammation, and the Host Response to Injury Large-Scale Collaborative Research, P. (2011). Dissecting inflammatory complications in critically injured patients by within-patient gene expression changes: a longitudinal clinical genomics study. PLoS Med 8, e1001093.
Dutton, R. P., Stansbury, L. G., Leone, S., Kramer, E., Hess, J. R., and Scalea, T. M. (2010). Trauma mortality in mature trauma systems: are we doing better? An analysis of trauma mortality patterns, 1997-2008. J Trauma 69, 620-626.
Fishman, P., Bar-Yehuda, S., Liang, B. T., and Jacobson, K. A. (2012).
Pharmacological and therapeutic effects of A3 adenosine receptor agonists. Drug Discov Today 17, 359-366.
Friedman, J., Hastie, T., and Tibshirani, R. (2010). Regularization Paths for Generalized Linear Models via Coordinate Descent. J Stat Softw 33, 1-22.
Georgiev, H., Ravens, I., Papadogianni, G., and Bernhardt, G. (2018). Coming of Age: CD96 Emerges as Modulator of Immune Responses. Front Immunol 9, 1072.
Glance, L. G., Stone, P. W., Mukamel, D. B., and Dick, A. W. (2011). Increases in mortality, length of stay, and cost associated with hospital-acquired infections in trauma patients. Arch Surg 146, 794-801.
Harris, V. C., Haak, B. W., Boele van Hensbroek, M., and Wiersinga, W. J. (2017). The Intestinal Microbiome in Infectious Diseases: The Clinical Relevance of a Rapidly Emerging Field. Open Forum Infect Dis 4, of x 144.
Hashmi, A., Ibrahim-Zada, I., Rhee, P., Aziz, H., Fain, M. J., Friese, R. S., and Joseph, B. (2014). Predictors of mortality in geriatric trauma patients: a systematic review and meta-analysis. J Trauma Acute Care Surg 76, 894-901.
Heffernan, D. S., Monaghan, S. F., Thakkar, R. K., Machan, J. T., Cioffi, W. G., and Ayala, A. (2012). Failure to normalize lymphopenia following trauma is associated with increased mortality, independent of the leukocytosis pattern. Crit Care 16, R12.
Heron, M. (2018). Deaths: Leading Causes for 2016. Natl Vital Stat Rep 67, 1-77.
Huang da, W., Sherman, B. T., and Lempicki, R. A. (2009). Systematic and integrative analysis of large gene lists using DAVID bioinformatics resources. Nat Protoc 4, 44-57.
Huber-Lang, M., Lambris, J. D., and Ward, P. A. (2018). Innate immune responses to trauma. Nat Immunol 19, 327-341.
Imamura, R., and Matsumoto, K. (2017). Hepatocyte growth factor in physiology and infectious diseases. Cytokine 98, 97-106.
Islam, M. N., Bradley, B. A., and Ceredig, R. (2016). Sterile post-traumatic immunosuppression. Clin Transl Immunology 5, e77.
Jamulitrat, S., Narong, M. N., and Thongpiyapoom, S. (2002). Trauma severity scoring systems as predictors of nosocomial infection. Infect Control Hosp Epidemiol 23, 268-273.
Kauffmann, A., Gentleman, R., and Huber, W. (2009). arrayQualityMetrics—a bioconductor package for quality assessment of microarray data. Bioinformatics 25, 415-416.
Knaus, W. A., Draper, E. A., Wagner, D. P., and Zimmerman, J. E. (1985). APACHE II: a severity of disease classification system. Crit Care Med 13, 818-829.
Kramer, A., Green, J., Pollard, J., Jr., and Tugendreich, S. (2014). Causal analysis approaches in Ingenuity Pathway Analysis. Bioinformatics 30, 523-530.
Krug, E. G., Sharma, G. K., and Lozano, R. (2000). The global burden of injuries. Am J Public Health 90, 523-526.
Kuhn, M. (2008). Building Predictive Models in R Using the caret Package. Journal of Statistical Software 28.
Lo, A., Chernoff, H., Zheng, T., and Lo, S. H. (2015). Why significant variables aren't automatically good predictors. Proc Natl Acad Sci USA 112, 13892-13897.
Lorenz, K. J., Schallert, R., and Daniel, V. (2015). Immunonutrition—the influence of early postoperative glutamine supplementation in enteral/parenteral nutrition on immune response, wound healing and length of hospital stay in multiple trauma patients and patients after extensive surgery. GMS Interdiscip Plast Reconstr Surg DGPW 4, Doc15.
Marshall, J. C. (2014). Why have clinical trials in sepsis failed? Trends Mol Med 20, 195-203.
Massachusetts, T. C.o. (2020). Crisis Standards of Care Planning Guidance for the COVID-19 Pandemic (Department of Public Health, Executive Office of Health and Human Services).
Medicine, I.o. (2013). Crisis Standards of Care: A Toolkit for Indicators and Triggers (Washington, D.C.: The National Academies Press).
Medicine, U.o.P. D.o.C. C. (2020). A Model Hospital Policy for Allocating Scarce Critical Care Resources.
Mitka, M. (2011). Drug for severe sepsis is withdrawn from market, fails to reduce mortality. JAMA 306, 2439-2440.
Osler, T., Glance, L. G., and Hosmer, D. W. (2010). Simplified estimates of the probability of death after burn injuries: extending and updating the baux score. J Trauma 68, 690-697.
Paladino, L., Subramanian, R. A., Bonilla, E., and Sinert, R. H. (2010). Leukocytosis as prognostic indicator of major injury. West J Emerg Med 11, 450-455.
Parikh, R. B., Kakad, M., and Bates, D. W. (2016). Integrating Predictive Analytics Into High-Value Care: The Dawn of Precision Delivery. JAMA 315, 651-652.
Riddell, E., and Vader, J. M. (2017). Potential Expanded Indications for Neprilysin Inhibitors. Curr Heart Fail Rep 14, 134-145.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47.
Robin, X., Turck, N., Hainard, A., Tiberti, N., Lisacek, F., Sanchez, J. C., and Muller, M. (2011). pROC: an open-source package for R and S+ to analyze and compare ROC curves. BMC Bioinformatics 12, 77.
Servant, N., Gravier, E., Gestraud, P., Laurent, C., Paccard, C., Biton, A., Brito, I., Mandel, J., Asselain, B., Barillot, E., et al. (2010). EMA—A R package for Easy Microarray data analysis. BMC Res Notes 3, 277.
Stevenson, M., Nunes, E., Heuer, C., Marshall, J., Sanchez, J., Thornton, R., Reiczigel, J., Robison-Cox, J., Sebastiani, P., Solymos, P., et al. (2019). epiR: Tools for the Analysis of Epidemiological Data. Available on the web at CRANR-projectorg/package=epiR.
Sweeney, T. E., Shidham, A., Wong, H. R., and Khatri, P. (2015). A comprehensive time-course-based multicohort analysis of sepsis and sterile inflammation reveals a robust diagnostic gene set. Sci Transl Med 7, 287ra271.
Sweeney, T. E., and Wong, H. R. (2016). Risk Stratification and Prognosis in Sepsis: What Have We Learned from Microarrays? Clin Chest Med 37, 209-218.
Tosh, P. K., and McDonald, L. C. (2012). Infection control in the multidrug-resistant era: tending the human microbiome. Clin Infect Dis 54, 707-713.
Ward, P. A. (2005). Immunosuppression after trauma. Crit Care Med 33, 1453-1454.
West, J. A., Davis, C. P., Sunwoo, H., Simon, M. D., Sadreyev, R. I., Wang, P. I.,
Tolstorukov, M. Y., and Kingston, R. E. (2014). The long noncoding RNAs NEAT1 and MALAT1 bind active chromatin sites. Mol Cell 55, 791-802.
Wu, J., and Irizarry, R. A. (2017). gcrma: Background Adjustment Using Sequence Information. R package version 2500.
Yan, S., Tsurumi, A., Que, Y. A., Ryan, C. M., Bandyopadhaya, A., Morgan, A. A., Flaherty, P. J., Tompkins, R. G., and Rahme, L. G. (2015). Prediction of multiple infections after severe burn trauma: a prospective cohort study. Ann Surg 261, 781-792.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of detecting or predicting risk of developing multiple independent infection episodes (MITE) in a subject who has experienced blunt trauma, the method comprising:
providing a sample comprising blood, serum, or plasma from a subject who has experienced blunt trauma;
determining a level of one, two, or more transcripts in a 15 probe set biomarker panel comprising or consisting of hepatocyte growth factor (HGF); kelch repeat and BTB domain containing 7 (KBTBD7); adenosine A3 receptor (ADORA3); ADP-ribosylation factor-like GTPase 4A (ARL4A); epiplakin 1 (EPPK1); zinc finger protein 354A (ZNF354A); SH3 and PX domains 2B (SH3PXD2B); RNase A family 1 (RNASE1), BTB domain containing 19 (BTBD19), zeta chain of T cell receptor associated protein kinase 70 kDa (ZAP70); endoplasmic reticulum aminopeptidase 2 (ERAP2); CD96 molecule (CD96); membrane metallo-endipeptidase (MME); killer cell lectin-like receptor subfamily F, member 1 (KLRF1); and/or non-coding transcript, nuclear paraspeckle assembly transcript 1 (NEAT1), and
(i) comparing the level of the transcript to a reference level, wherein the presence of: an increase in one or more of: hepatocyte growth factor (HGF); kelch repeat and BTB domain containing 7 (KBTBD7); adenosine A3 receptor (ADORA3); ADP-ribosylation factor-like GTPase 4A (ARL4A); epiplakin 1 (EPPK1); zinc finger protein 354A (ZNF354A); SH3 and PX domains 2B (SH3PXD2B); RNase A family 1 (RNASE1), or BTB domain containing 19 (BTBD19), or
a decrease in one or more of: zeta chain of T cell receptor associated protein kinase 70 kDa (ZAP70); endoplasmic reticulum aminopeptidase 2 (ERAP2); CD96 molecule (CD96); membrane metallo-endipeptidase (MME); killer cell lectin-like receptor subfamily F, member 1 (KLRF1); and/or non-coding transcript, nuclear paraspeckle assembly transcript 1 (NEAT1),
indicates that the subject has, or has an increased risk of developing, MIIE; or (ii) calculating a score based on the transcript levels, and comparing the score to a threshold or reference score, wherein a score about the threshold or reference score indicates that the subject has, or has an increased risk of developing, MIIE.

2. The method of claim 1, comprising determining a level of all of ZNF354A; EPPK1; RNASE1; BTBD19; ADORA3; KBTBD7; SH3PXD2B; CD96; ARL4A; ZAP70; ERAP2; HGF; KLRF1; NEAT1; and MME.

3. The method of claim 1, further comprising determining a level of one, two, or more of Interleukin 1 receptor, type II (IL1R2) and mannose receptor, C type 1 (MRC1); importin 11 (IPO11); dedicator of cytokinesis 4 (DOCK4); Kruppel-like factor 9 (KLF9); nebulette (NEBL); a different probe set for MME; ribosomal protein S6 kinase, 90 kDa, polypeptide 5 (RPS6KA5); killer cell lectin-like receptor, subfamily K, member 1 (KLRK1); granzyme K (GZMK); and/or ADP-ribosylation factor-like GTPase 4C (ARL4C).

4. The method of claim 3, comprising determining a level of ARL4C; KLRK1; SH3PXD2B; EPPK1; ZNF354A; RNASE1; MME; BTBD19; MRC1; ADORA3; NEBL; KBTBD7; IPO11; RPS6KA5; KLF9; DOCK4; IL1R2; CD96; ARL4A; ERAP2; GZMK; NEAT1; KLRF1; HGF; MME; and ZAP70.

5. The method of claim 3, wherein:
an increase in one or more of interleukin 1 receptor, type II (IL1R2); mannose receptor, C type 1 (MRC1); importin 11 (IPO11); dedicator of cytokinesis 4 (DOCK4); Kruppel-like factor 9 (KLF9); and/or nebulette (NEBL), or
a decrease in MME, ribosomal protein S6 kinase, 90 kDa, polypeptide 5 (RPS6KA5);
killer cell lectin-like receptor, subfamily K, member 1 (KLRK1); granzyme K (GZMK); and/or ADP-ribosylation factor-like GTPase 4C (ARL4C) indicates that the subject has, or has an increased risk of developing, an infection.

6. The method of claim 2, wherein the infections include infection with a Gram positive or Gram negative bacterium, virus, or fungus, optionally selected from Streptococcus pneumonia, Streptococcus viridans, Staphylococcus aureus, Enterococcus species, Coagulase negative staphylococci, and Streptococcus pneumoniae and viridian, Clostridium sp.; Candida species, Enterobacter species, Acinetobacter species, Pseudomonas aeruginosa, Haemophilus influenza, Bacteroides species, Klebsiella pneumoniae, Neisseria, Proteus, Serratia marcescens, Escherichia coli, Stenotrophomonas, and Candida species.

7. The method of claim 1, further comprising treating the subject, optionally by administering a broad-spectrum antibiotic, increasing the frequency or length of monitoring of the subject for infection; implementing prophylactic measures; and/or enhancing patient nutrition.

8. The methods of claim 1, wherein the subject is a mammal, preferably a human.

9. A method comprising:
providing a sample comprising blood, serum, or plasma from a subject who has experienced blunt trauma;
detecting a level of transcripts consisting of two, three, or more of hepatocyte growth factor (HGF); kelch repeat and BTB domain containing 7 (KBTBD7); adenosine A3 receptor (ADORA3); ADP-ribosylation factor-like GTPase 4A (ARL4A); epiplakin 1 (EPPK1); zinc finger protein 354A (ZNF354A); SH3 and PX domains 2B (SH3PXD2B); RNase A family 1 (RNASE1), BTB domain containing 19 (BTBD19), zeta chain of T cell receptor associated protein kinase 70 kDa (ZAP70); endoplasmic reticulum aminopeptidase 2 (ERAP2); CD96 molecule (CD96); membrane metallo-endipeptidase (MME); killer cell lectin-like receptor subfamily F, member 1 (KLRF1); and non-coding transcript, nuclear paraspeckle assembly transcript 1 (NEAT1).

10. The method of claim 9, comprising determining a level of all of ZNF354A; EPPK1; RNASE1; BTBD19; ADORA3; KBTBD7; SH3PXD2B; CD96; ARL4A; ZAP70; ERAP2; HGF; KLRF1; NEAT1; and MME.

11. The method of claim 9, further comprising determining a level of one or more transcripts selected from the group consisting of Interleukin 1 receptor, type II (IL1R2) and mannose receptor, C type 1 (MRC1); importin 11 (IPO11); dedicator of cytokinesis 4 (DOCK4); Kruppel-like factor 9 (KLF9); nebulette (NEBL); a different probe set for MME; ribosomal protein S6 kinase, 90 kDa, polypeptide 5 (RPS6KA5); killer cell lectin-like receptor, subfamily K, member 1 (KLRK1); granzyme K (GZMK); and ADP-ribosylation factor-like GTPase 4C (ARL4C).

12. The method of claim 11, comprising determining a level of ARL4C; KLRK1; SH3PXD2B; EPPK1; ZNF354A; RNASE1; MME; BTBD19; MRC1; ADORA3; NEBL; KBTBD7; IPO11; RPS6KA5; KLF9; DOCK4; IL1R2; CD96; ARL4A; ERAP2; GZMK; NEAT1; KLRF1; HGF; MME; and ZAP70.

13. The method of claim 1, further comprising calculating a score based on the levels.

14. The method of claim 13, wherein the score is calculated using an algorithm comprising summation or weighted summation of normalized levels of the biomarkers.

15. The method of claim 13, wherein the score is calculated using principal components analysis (PCA), linear regression, support vector machine (SVM), decision trees, K-nearest neighbors (KNN), K-means, gradient boosting, or random forest methods.

16. The method of claim 1, wherein determining a level comprises using PCR-based methods RNA sequencing, next generation sequencing, digital gene expression analysis, or microarray analysis.

17. The method of claim 1, wherein the sample is obtained from the subject within 48 hours of the blunt trauma.

18. The method of claim 1, wherein the subject does not have clinical signs of an infection when the sample is obtained.
